(19) United States
(12) Patent Application Publication
(10) Pub. No.: US 2020/0265946 A1
(43) Pub. Date: Aug. 20, 2020

(54) MEDICAL IMAGE WORKFLOW SYSTEM AND METHOD

(71) Applicant: ALGOTEC SYSTEMS LTD., Ra,anana, IL (US)

(72) Inventors: Menashe BENJAMIN, Neve Afek Rosh Ha'ain (IL); Meir COHEN, Givat Shmuel (IL); Ron KIMCHI, Tel Aviv (IL); Alex AISEN, Carmel, IN (US)

(21) Appl. No.: 16/306,704

(22) PCT Filed: Jun. 15, 2017
(86) PCT No.: PCT/US2017/037678

§ 371 Date: Dec. 3, 2018

Related U.S. Application Data
(60) Provisional application No. 62/351,495, filed on Jun. 17, 2016.

(51) Int. Cl. G16H 40/20; G06K 9/00

(52) U.S. Cl. CPC G16H 40/20; G06K 9/00469

(57) ABSTRACT

A method of automatically assigning each of a plurality of medical imaging cases for reading by one of a plurality of readers, comprising: receiving the medical imaging cases; listing each case, at different times, on worklists displayed to one or more readers who are allowed to choose the case for reading at that time; receiving information on choices of cases by the readers; and assigning cases to readers who choose them; wherein for at least some of the cases, initially only a portion of the readers are allowed to choose the case, but over time, as the case becomes more urgent to read, the case is escalated a first time by adding one or more other readers who are allowed to choose the case, and over further time, if the case is still not read, the case is escalated at least one additional time.

### CROSS REFERENCE TO RELATED APPLICATION/S

This application claims benefit under 35 U.S.C. 119(e) of U.S. Provisional Patent Application No. 62/351,495, filed Jun. 17, 2016, entitled “RADIOLOGY WORKFLOW SYSTEM AND METHOD,” in the names of Benjamin et al. The contents of the above application are incorporated by reference as if fully set forth herein in their entirety.

### FIELD AND BACKGROUND OF THE INVENTION

The present invention, in some embodiments thereof, relates to a method and system for providing cases involving medical images to readers for reading and, more particularly, but not exclusively, to a computerized system that provides multiple types of radiology cases, coming from multiple sites, to radiologists for reading. Medical imaging is an important component of medical care, and involves performing imaging procedures on patients using equipment such as conventional x-rays, CT scans, ultrasound scans, MRI scans, and nuclear medicine studies. The scans are acquired from patients using different types of imaging equipment, termed “modalities.” The images acquired are then interpreted, that is the normal and abnormal findings are identified, and possible diagnoses are enumerated. The interpretation process is usually the province of the medical specialty of radiology, and the studies are interpreted by radiologists. Sites such as hospitals and other medical facilities may generate a large number of medical imaging cases of different types, and assign these cases to many different radiologists to read.

Years ago, most radiologists could interpret most varieties of imaging studies. Groups of radiologists would typically work at hospitals or imaging centers, and interpret all imaging studies performed. The images were usually recorded on special photographic film, and presented to radiologists to interpret through use of illuminated mechanical devices that could accommodate multiple studies, called alternators.

This is no longer the case. In recent years, the medical specialty of radiology has become divided into subspecialties, often based on the region of the body being imaged. For example, images of the brain and the spine are commonly interpreted by subspecialist radiologists called neuro-radiologists. Other subspecialties include, among others, body radiology, chest radiology, mammography, pediatric radiology, and musculoskeletal radiology. In addition, the types of equipment used to acquire medical imaging studies have become more sophisticated. As noted above, imaging studies are acquired on a variety of devices, including plain x-ray equipment, CT scanners, MR scanners, ultrasound devices, and nuclear medicine devices.

Additionally, there have been substantial changes in the technology used by radiologists to interpret studies. Today, almost all images are stored and distributed as electronic data, and viewed on computer workstations. The use of film is viewed by some as expensive and not practical, as some studies are comprised of hundreds or even thousands of individual images. The same information technology also allows cases to be transmitted from one location to another over data networks. The computer systems for receiving, storing, transferring, and displaying radiology studies so radiologists can interpret them are referred to as Picture Archiving and Communication Systems, or PACS.

Previously, relatively small groups of radiologists provided professional services at single hospitals. Radiologists would usually interpret the imaging studies in the order in which they were performed, with exceptions such as for urgent studies, which would be given priority. Today, the situation has become more complicated. Studies take longer to interpret, so prioritization has become more important. As radiologists have become more specialized, it becomes desirable for subspecialty radiologists to interpret studies matched to their specific training. In addition, the work environment of radiology has changed; the use of medical imaging has increased, and radiologists may be expected to read studies faster and more efficiently than they were before.

Radiology groups have grown substantially in size, and cases are often transmitted between hospitals and imaging facilities, and interpreted in one or more centralized locations. It may be advantageous for cases to be interpreted by radiologists physically located at or affiliated with the hospitals or facilities at which the cases originated. Also, as radiology groups have grown in size, it may be desirable for workloads to be managed in a manner that is efficient, while providing an equitable distribution of work. Managing this complex work environment has become challenging. Workflow management can involve the balancing of often conflicting requirements, such as prioritizing the most urgent cases, making use of subspecialty skills, maintaining relationships between radiologists and the hospitals and referring physicians they serve, and distributing the workload among many radiologists in a fair and equitable manner.

To help in managing this work environment, many commercial PACS systems now include simple functionality for creating worklists of cases to be interpreted. These worklists can be filtered by the radiologists on the basis of criteria such as the location at which the studies were acquired, the imaging modality used, and the type of study. The radiologists may also have the ability to sort the cases in the worklist, based on the value of one or more parameters, for example based on when the image was made, or based on urgency of reading the image, if the urgency is indicated in metadata that comes with the image, for example if urgent cases are flagged in the metadata as “STAT.”

As described in <https://www.crunchbase.com/organization/clario-medical-imaging#/entity> and <https://www.clariomedical.com/radiology-worklist-solutions/smart-worklist-private-practice/>, both downloaded on Feb. 21, 2017, Clario Medical provides radiology workflow software solutions to hospitals, imaging centers, and private practice radiology groups.

U.S. 2015/0347693 (Lam et al) describes a computer-implemented method for selecting readers for analyzing a medical image.

U.S. 2013/0132105 and U.S. 2013/0132142, both to Wood-Salomon et al, describe assigning a radiology work study such as a radiology image study to be read within a radiology work list associated with at least one radiologist.

U.S. 2008/0140454 (Hernandez et al) describes methods and systems for automating and managing efficient workflow for the viewing and analysis of diagnostic images within a healthcare enterprise network.

Reference is also made to: U.S. 2014/0142969 (Backhaus et al), U.S. 2014/0142983 (Backhaus et al), U.S. 2014/0088987 (Backhaus), U.S. Pat. No. 7,756,724 (Gropper et al), and U.S. 2006/0109961 (Mahesh et al).

### SUMMARY OF THE INVENTION

An aspect of some embodiments of the invention concerns a method for assigning medical imaging cases to readers for reading, for example assigning radiology cases to radiologists, using a computerized system, in which only certain cases are exposed (that is, made visible) to each reader to choose for reading, and each reader is only encouraged to choose certain of those cases. As time goes on without a given case being read, that case becomes more urgent for reading, and more readers are exposed to it and/or encouraged to choose it. A convenient user interface may provide a single list of all the cases available to a given reader, as well as listing the number of unread cases in each subspecialty and/or from each site of origin. Readers who engage in “cherry picking,” choosing too many lower priority cases while avoiding higher priority cases without a good reason, may be discouraged by the system from doing so.

There is thus provided, in accordance with an exemplary embodiment of the invention, a method of automatically assigning each of a plurality of medical imaging cases for reading by one of a plurality of readers, the method comprising: a) receiving the medical imaging cases by a computer system, from one or more sites; b) the computer system including information about each case, at different times, on worklists that the computer system displays on workstations of one or more readers who are allowed to choose the case for reading at that time, the worklists indicating to each of those readers if they are encouraged to choose the case for reading at that time; c) the computer system receiving information on choices of cases by the readers; and d) the computer system assigning cases to workstations of readers who choose them; wherein for at least some of the cases, initially only a portion of the readers are allowed or encouraged to choose the case, but over time, as the case becomes more urgent to read if it is still not read, the case is escalated a first time by the computer system adding one or more other readers to the readers who are allowed or encouraged to choose the case, and over further time, if the case is still not read, the case is escalated at least one additional time by the computer system adding one or more other readers to the readers who are allowed or encouraged to choose the case.

Optionally, the medical imaging cases comprise radiology cases and the readers who are allowed to choose the radiology cases are radiologists.

Optionally, each reader has a profile accessible to the computer system indicating the reader's characteristics, and initially, readers are allowed to choose a case, or are encouraged to choose a case, only if they there is a subspecialty which matches the case indicated on the reader's profile, or only if there is a relationship with a site of origin of the case indicated on the reader's profile, or both, and when the case is escalated, the added readers have a different subspecialty, or do not have a relationship with the site of origin, or both, indicated on their profiles.

Optionally, when a case is escalated one of the times, there is no relationship with the site of origin indicated on the added readers' profiles, but there is a subspecialty which matches the case indicated on the added readers' profiles, and when the case is escalated at a later one of the times, there is no subspecialty which matches the case indicated on the added readers' profiles.

Optionally, the medical imaging cases comprise one or more of pathology cases, dermatology cases, gastroenterology cases, pulmonology cases, and ophthalmology cases, and the readers who are allowed to choose the medical imaging cases are medical personnel with corresponding specialties.

Optionally, the method also comprises: a) for each case, the computer system determining an urgency for reading the case as a function of time; b) for each case, the computer system determining one or more criteria for which of the readers will be allowed to choose the case for reading, and for which readers will be encouraged to choose the case for reading, as a function of time, based at least in part on the urgency for reading the case as a function of time; and including information on each case on worklists comprises including information on each case on worklists displayed on the workstations of the readers who are allowed to choose it at that time according to the criteria, indicating to the readers if they are encouraged to choose the case at that time according to the criteria.

Optionally, determining criteria for which readers will be encouraged to choose a case at a given time comprises determining criteria for setting a relative priority of a case that a reader is allowed to choose, compared to other cases that the reader is allowed to choose, based at least in part on the urgency for reading the case at that time, and including information about each case on worklists of one or more readers who are allowed to choose it comprises indicating on the worklist of each of those readers the relative priority of the case.

Optionally, determining criteria for determining a relative priority of a case for a reader comprises determining criteria for whether or not the case is recommended for the reader.

Optionally, each reader has a profile accessible to the computer system indicating the reader's characteristics, and the criteria depend on whether or not a reader's profile indicates an association with the site that the case was received from.

Optionally, each reader has a profile accessible to the computer system indicating the reader's characteristics, and the criteria depend on whether or not a reader's profile indicates a subspecialty that matches the case.

Optionally, determining an urgency for reading a case comprises determining a deadline for reading the case, and the urgency for reading the case depends at least in part on how much time remains before the deadline.

Optionally, determining criteria comprises determining criteria that depend on one or more of work shift, time of day, day of the week, and whether it is a holiday.

In an embodiment of the invention, the method also comprises analyzing images from the medical imaging cases by imaging analytics circuitry to determine a presence or absence of one or more features that indicate a higher degree of urgency for reading the case, wherein determining an urgency for reading the case as a function of time comprises determining a higher degree of urgency at a given time for a case in which the one or more features are present than for a case with otherwise identical characteristics received at the same time for which the one or more features are absent.

Optionally, according to the criteria, for at least some times and at least some characteristics of cases, more readers would be allowed or encouraged to choose a case in which the one or more features are present, than a case with otherwise identical characteristics received at the same time, in which the one or more features are absent.

Optionally, the one or more features comprise free air in a patient's abdominal cavity or thorax.

Additionally or alternatively, the medical imaging cases comprise screening studies of a population of apparently healthy patients for a medical condition that only occurs in a small minority of the population of patients being screened, and the one or more features indicate a presence of the medical condition.

Optionally, the cases received pertain to a plurality of different medical conditions, and determining an urgency for reading the case comprises determining an urgency that depends at least in part on the medical condition that the case pertains to.

In an exemplary embodiment of the invention, including information about each case on worklists displayed on workstations of one or more readers comprises including information about a case that was escalated on a same worklist on a workstation of one of the readers who was added when the case was escalated, as information about a case that was not escalated.

Optionally, the method comprises the computer system determining a single order of priority for all cases for which information is included on the worklist.

Alternatively, the method comprises the computer system arranging cases for which information is included on the worklist into two or more sub-worklists, each sub-worklist including a different category of cases, and determining a separate order of priority for each sub-worklist.

In an embodiment of the invention, each reader has a profile indicating the reader's characteristics, accessible to the computer system, the method also comprising the computer system displaying together with the worklist on the workstations of at least some of the readers how many unread cases match each of a plurality of different subspecialties, including subspecialties that are not indicated on the profile of that reader, or how many unread cases come from each of a plurality of different sites of origin, or both.

Optionally, displaying together with the worklist comprises the computer system displaying on the workstations of at least some of the readers how many unread cases come from each of a plurality of sites of origin.

Optionally, displaying together with the worklist comprises the computer system displaying on the workstations of at least some of the readers how many unread cases match each of a plurality of different subspecialties, including subspecialties that are not indicated on the profile of that reader.

Optionally, displaying how many unread cases match each of a plurality of different subspecialties comprises at least displaying how many cases from each of a plurality of sites of origin match each of a plurality of subspecialties.

Optionally, the method also includes: a) assessing, from the display of how many unread cases match each of a plurality of different subspecialties, that too large a backlog of unread cases is building up for one of the subspecialties; and b) increasing a number of readers available for reading cases matching that subspecialty, encouraging an increase in a speed at which readers read cases matching that subspecialty, or both.

Optionally, the method also comprises the computer system displaying together with the worklist of at least some of the readers how many unread cases belong to each of a plurality of different categories of levels of urgency.

There is further provided, according to an exemplary embodiment of the invention, a method of automatically assigning each of a plurality of medical imaging cases for reading by one of a plurality of readers, the method comprising: a) receiving the medical imaging cases by a computer system; b) for each case, the computer system determining an urgency for reading the case as a function of time; c) for each case, the computer system determining one or more criteria for which of the readers will be allowed to choose the case for reading, and for each reader who is allowed to choose the case, the computer system determining a relative priority of the case compared to other cases the reader is allowed to choose, based at least in part on the urgency for reading the case; d) the computer system including information about each case in a worklist that the computer system displays on a workstation of each of one or more readers who are allowed to choose it for reading, the information indicating to the readers the relative priority of different cases they are allowed to choose; e) the computer system receiving information on choices of cases by the readers; f) the computer system assigning cases to readers who choose them; and g) the computer system keeping track of a measure of an extent to which different readers choose cases of lower priority than other cases they are allowed to choose.

Optionally, determining an urgency for reading the case comprises assigning the case to a category according to one or more characteristics of the case, and determining a relative priority of the case compared to other cases the reader is allowed to choose comprises determining an order of priority of the case compared to other cases in a same category as the case.

Optionally, the method also comprises the computer system receiving reasons provided by readers who choose cases with lower priority than other cases they are allowed to choose, explaining why they chose those cases.

Optionally, the method comprises instituting sanctions against readers who choose cases more than a threshold number of times in a specified time interval or while choosing a specified number of cases, with lower priority than other cases they are allowed to choose, without providing approved reasons.

Optionally, the method also comprises the computer system receiving reasons from readers who choose cases with lower priority than other cases they are allowed to choose, wherein the sanctions are not applied to readers who have provided approved reasons for choosing the cases with lower priority.

Optionally, the sanctions comprise the computer system making it known to other readers that a reader has frequently chosen cases with lower priority than other cases the reader was allowed to choose.

Optionally, the sanctions comprise the computer system allowing the readers to choose only less desirable cases than they would be allowed to choose if they were not sanctioned, at least for a period of time.

Optionally, determining a relative priority of the case compared to other cases comprises making a binary decision about whether the case is recommended, or not recommended, for the reader.

Optionally, the method comprises the computer system providing an initial value for an integer N before displaying information about any case, and determining a relative priority of the case compared to other cases comprises determining a relative priority of the case at least compared to other cases for which information is displayed to that reader in a same category as the case, and making a binary decision comprises deciding that up to the N highest priority cases in that category for which information is displayed to that reader are recommended and any other cases in that category for which information is displayed to that reader are not recommended.

Optionally, the method also comprises the computer system evaluating one or more measures of performance of reading the cases, using the measures to determine a new value of N that is expected to improve an overall level of the performance, and then using the new value of N for displaying information about each case in that category.

Optionally, determining a new value of N comprises determining the new value of N from the measures of performance using a fixed algorithm.

Optionally, the information included in the worklist indicates to a reader a relative priority of the different cases that the reader is allowed to choose for reading.

Optionally, for at least some of the cases, initially only a portion of the readers are allowed or encouraged to choose the case, but over time, as the case becomes more urgent to read if it is still not read, one or more other readers are added by the computer system to the readers who are allowed or encouraged to choose the case.

In an embodiment of the invention, determining an urgency for reading each case comprises using a rule that depends on one or more characteristics of the case, and determining the criteria comprises determining criteria that depend on characteristics of a case, characteristics of a reader, and the urgency for reading a case, the method also comprising: a) the computer system evaluating one or more measures of performance of reading the cases; b) the computer system using the measures of performance to determine a new value of one or more parameters defining the rule or the criteria that is predicted to improve an overall level of the performance; and c) the computer system applying the new value of the one or more parameters to adjust the rule or the criteria for new medical imaging cases received by the computer.

Optionally, evaluating one or more measures of performance is repeated at least once to obtain new values of the one or more measures of performance, after applying the new value of the one or more parameters to adjust the rule or criteria, and using the measures of performance to determine a new value of the one or more parameters and applying the new value are repeated at least once using at least the new values of the one or more measures of performance.

Optionally, using at least the new measures of performance comprises using the measures of performance evaluated over at least two different time periods, and values of the one or more parameters during those two different time periods, to determine the new value of the one or more parameters, according to a feedback algorithm.

Optionally, using the measures of performance to determine a new value comprises determining the new value from the measures of performance using a fixed algorithm.

Optionally, using a rule to determine an urgency for reading a case comprises using the rule to determine a deadline for reading the case, setting the criteria comprises allowing or encouraging additional readers to choose the case, or giving the case relative priority over more other cases for some readers, or both, after an escalation time that depends on the deadline in a way specified by the criteria, and the new value of one or more parameters changes the escalation time by adjusting how the escalation time depends on the deadline.

Optionally, the criteria for which of the readers will be allowed to choose a case and for which of the readers will be encouraged to choose a case impose a maximum number of cases N that a reader is allowed to choose from or that a reader is encouraged to choose from, for at least one category of cases and at least some readers, and the new value of one or more parameters comprises a new value of N, for at least that category of cases and those readers.

Optionally, determining the relative priority comprises determining an order of priority of cases, for at least that category and at least those readers, and the criteria only allow those readers to choose, or only encourage those readers to choose, one of the N highest cases in order of priority.

Optionally, the criteria impose a same or different number of cases N for each of a plurality of different categories of cases, and the change in N for each category is determined by measures of performance that are specific to individual categories.

Optionally, the measures of performance each comprise a portion, between 0% and 100% inclusive, of cases in one category that have not been read by the deadline or by a specified time interval before or after the deadline.

Optionally, the method also comprises displaying, at least to those readers, the measures of performance, specific to individual categories, that determine the new value of N for each category.

Optionally, the method comprises reducing N for a first category, when the measures of performance show better performance for the first category than for a second category.

In an embodiment of the invention, determining a relative priority of the case compared to other cases the reader is allowed to choose comprises setting an order of priority of the cases the reader is allowed to choose at a given time, by: a) the computer system providing a deadline for reading each case; b) the computer system finding the time to deadline for each case, at the given time; c) the computer system providing an index of importance function of time to deadline and of characteristics of a case; d) the computer system calculating an index of importance for each case according to the index of importance function; and e) the computer system setting the order of priority, the order of priority being higher for cases with higher index of importance.

Optionally, for a given set of characteristics the index of importance function increases or remains the same with decreasing time to deadline, and has a plurality of different values for the times to deadline for which it is defined.

Optionally, the method also comprises classifying each case to one of two or more categories corresponding to different levels of urgency for reading the cases, wherein the index of importance function depends on one or more characteristics of the case at least including the category, the index of importance function being different for cases in different categories but with the same time to deadline and other characteristics, for at least some categories.

Optionally, for two different categories corresponding to a higher and a lower level of urgency, for a given time to deadline and other characteristics, the index of importance function is higher for the category corresponding to the higher level of urgency than for the category corresponding to the lower level of urgency.

Optionally, for at least a first set and a second set of characteristics of cases: a) the index of importance function is a first function of time to deadline for the first set, and is a second function of time to deadline for the second set; b) a smallest interval that includes all values of the first function overlaps a smallest interval that includes all values of the second function; c) the first function and the second function have different values for at least one time to deadline for which they are both defined; and d) when the second function is shifted in time to deadline by any constant shift, then the first function and the shifted second function have different values for at least one time to deadline for which they are both defined, if there is any time to deadline for which they are both defined for that shift.

Optionally, the first function and second function increase or remain unchanged for decreasing time to deadline, over the times to deadline for which they are defined, and the first function and the second function each have a plurality of different values, over the times to deadline for which they are defined.

Optionally, for at least one set of characteristics of cases, the index of importance function is a non-monotonic function of time to deadline.

There is further provided, in accordance with an exemplary embodiment of the invention, a computer system for automatically assigning each of a plurality of medical imaging cases for reading by one of a plurality of readers, the system comprising: a) a receiving module configured to receive medical imaging cases from one or more sites; b) a workstation used by each reader; c) a reader I/O module configured to include information about different sets of the received medical imaging cases in worklists that the computer system displays on the workstations of different readers, to indicate that a reader is encouraged to choose a case for which information is displayed, to receive information about the readers' choices of cases for reading, and to assign cases to the workstations of the readers; and d) a computer running software configured to: 1) include information about each case, at different times, on worklists that the computer system displays on the workstations of one or more readers who are allowed to choose it for reading at that time, the information indicating to each of those readers if they are encouraged to choose the case for reading at that time; 2) receive information about choices of cases by the readers; and 3) assign cases to the workstations of readers who choose them; wherein the software is configured so that, for at least some of the cases, initially only a portion of the readers are allowed or encouraged to choose the case, but over time, as the case becomes more urgent to read if it is still not read, the case is escalated a first time by the computer system adding one or more other readers to the readers who are allowed or encouraged to choose the case, and over further time, if the case is still not read, the case is escalated at least one additional time by the computer system adding one or more other readers to the readers who are allowed or encouraged to choose the case.

There is further provided, in accordance with an exemplary embodiment of the invention, a computer system for automatically assigning each of a plurality of medical imaging cases for reading by one of a plurality of readers, the system comprising: a) a receiving module configured to receive medical imaging cases from one or more sites; b) a plurality of workstations, each used by one of the readers; c) a reader I/O module configured to display on a worklist on a workstation of each reader, information about a set of cases that the reader is allowed to choose for reading, the information indicating that the reader is encouraged to preferentially choose a displayed case over another displayed case, to receive information about the readers' choices of cases for reading, and to assign cases to the workstations of the readers; and d) a computer running software configured to: 1) for each case, determine an urgency for reading the case as a function of time; 2) for each case, determine one or more criteria for which of the readers will be allowed to choose the case for reading, and for each reader who is allowed to choose the case, determine a relative priority of the case compared to other cases the reader is allowed to choose, based at least in part on the urgency for reading the case; 3) include information about each case in worklists that the computer system displays on the workstations of one or more readers who are allowed to choose it for reading, the information indicating to each reader the relative priority of the case compared to other cases the reader is allowed to choose; 4) receive information about choices of cases by the readers; 5) assign cases to workstations of readers who choose them; and 6) keep track of a measure of an extent to which different readers choose cases of lower priority than other cases they are allowed to choose.

There is further provided, in accordance with an exemplary embodiment of the invention, a method of automatically assigning each of a plurality of medical imaging cases for reading by one of a plurality of readers, the method comprising: a) receiving the medical imaging cases by a computer system; b) for each case, the computer system determining an urgency for reading the case as a function of time, using a rule that depends on one or more characteristics of the case; c) for each case, the computer system determining one or more criteria, depending on characteristics of the case, characteristics of readers, and the urgency for reading the case, for which of the readers will be allowed to choose the case for reading, and for each reader who is allowed to choose the case, whether the reader is encouraged to choose the case, and any priority the case is given for each reader who is allowed to choose the case, over other cases the reader is allowed to choose; d) the computer system including information about each case in a worklist that the computer system displays on a workstation of each of one or more readers who are allowed to choose it for reading, the information indicating to the readers any relative priority of different cases they are allowed to choose; e) the computer system receiving information on choices of cases by the readers; f) the computer system assigning cases to readers who choose them; g) the computer system evaluating one or more measures of performance of reading the cases; h) the computer system using the measures of performance to determine a new value of one or more parameters defining the rule or the criteria that is predicted to improve an overall level of the performance; and i) the computer system applying the new value of the one or more parameters to adjust the rule or the criteria for new medical imaging cases received by the computer.

There is further provided, in accordance with an exemplary embodiment of the invention, a method of automatically assigning each of a plurality of medical imaging cases for reading by one of a plurality of readers, the method comprising: a) receiving the medical imaging cases by a computer system, from one or more sites; b) analyzing images from the medical imaging cases by imaging analytics circuitry to determine a presence or absence of one or more features that indicate a higher degree of urgency for reading the case; c) for each case, the computer system determining an urgency for reading the case as a function of time, including determining a higher degree of urgency at a given time for a case in which the one or more features are present than for a case with otherwise identical characteristics received at the same time for which the one or more features are absent; d) for each case, the computer system determining one or more criteria for which of the readers will be allowed to choose the case for reading, and for which readers will be encouraged to choose the case for reading, as a function of time, based at least in part on the urgency for reading the case as a function of time; e) the computer system including information about each case, at different times until it is chosen for reading, on worklists that the computer system displays on workstations of one or more readers who are allowed to choose it for reading at that time according to the criteria, the worklists indicating to each of those readers if they are encouraged to choose the case for reading at that time according to the criteria; f) the computer system receiving information on choices of cases by the readers; and g) the computer system assigning cases to workstations of readers who choose them.

There is further provided, in accordance with an exemplary embodiment of the invention, a method of automatically assigning each of a plurality of medical imaging cases for reading by one of a plurality of readers, the method comprising: a) receiving the medical imaging cases by a computer system, from one or more sites; b) the computer system including information about each case, at different times until it is chosen for reading, on worklists that the computer system displays on workstations of one or more readers who are allowed to choose it for reading at that time, the worklists indicating to each of those readers if they are encouraged to choose the case for reading at that time; c) the computer system receiving information on choices of cases by the readers; and d) the computer system assigning cases to workstations of readers who choose them; wherein for at least some of the cases, initially only a portion of the readers are allowed or encouraged to choose the case, but over time, as the case becomes more urgent to read if it is still not read, the case is escalated by the computer system adding one or more other readers to the readers who are allowed or encouraged to choose the case, the information about a case that was escalated being included on a same worklist on a workstation of one of the readers who was added, as information about a case that was not escalated.

There is further provided, in accordance with an exemplary embodiment of the invention, a method of automatically assigning each of a plurality of medical imaging cases for reading by one of a plurality of readers, each reader having a profile indicating the reader's characteristics, the method comprising: a) receiving the medical imaging cases by a computer system, from one or more sites; b) the computer system including information about each case, at different times until it is chosen for reading, on worklists that the computer system displays on workstations of one or more readers who are allowed to choose it for reading at that time, the worklists indicating to each of those readers if they are encouraged to choose the case for reading at that time; c) the computer system displaying together with the worklist on the workstations of at least some of the readers how many unread cases match each of a plurality of different subspecialties, including subspecialties that are not indicated on the profile of that reader, or how many unread cases come from each of a plurality of different sites of origin, or both; d) the computer system receiving information on choices of cases by the readers; and e) the computer system assigning cases to workstations of readers who choose them.

There is further provided, in accordance with an exemplary embodiment of the invention, a method of setting an order of priority, at a given time, for reading a plurality of medical imaging cases, comprising: a) the computer system providing a deadline for reading each case; b) the computer system finding the time to deadline for each case, at the given time; c) the computer system providing an index of importance function of time to deadline and of characteristics of a case; d) the computer system calculating an index of importance for each case according to the index of importance function; and e) the computer system setting the order of priority, the order of priority being higher for cases with higher index of importance.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as adjusting exposure parameters in response to measures of performance, and looking for features in a medical image that require immediate treatment, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

The foregoing and other objects, features, and advantages of the invention will be apparent from the following more particular description of the embodiments of the invention, as illustrated in the accompanying drawings.

The elements of the drawings are not necessarily to scale relative to each other. In the drawings:

FIG. 1 is a block diagram showing elements of a system for receiving radiology cases and assigning them to radiologists, and showing communication links between the elements, according to an exemplary embodiment of the invention;

FIG. 2 is a flowchart for a method of receiving radiology cases and assigning them to radiologists, which can be performed by the system shown in FIG. 1, according to an exemplary embodiment of the invention;

FIG. 3 is an exemplary schematic view of a display that could be seen by a radiologist when the method of FIG. 2 is performed, according to an exemplary embodiment of the invention;

FIG. 4 is a plot of an index of importance as a function of time to deadline for cases in five different categories, used to make a combined order of priority for reading cases in different categories, according to an exemplary embodiment of the invention;

FIG. 5 is a flowchart for a method of improving performance by adjusting parameters that determine which cases are exposed to which radiologists at each time, using feedback from one or more measures of performance, according to an exemplary embodiment of the invention; and

FIG. 6 is a flowchart for a method of using imaging analytics to flag certain radiology cases as having a higher urgency for reading, according to an exemplary embodiment of the invention.

### DESCRIPTION OF SPECIFIC EMBODIMENTS OF THE INVENTION

The following is a detailed description of some preferred embodiments, reference being made to the drawings in which the same reference numerals identify the same elements of structure in each of the several figures.

The present invention, in some embodiments thereof, relates to a method and system for providing cases involving medical images to workstations of readers for reading and, more particularly, but not exclusively, to a computerized system that provides multiple types of radiology cases, coming from multiple sites, to workstations of radiologists for reading.

Another object of some embodiments of the present disclosure is to promote interpretation of imaging studies by radiologists or other medical specialists, located at or affiliated with hospitals or imaging facilities where the imaging studies were done.

A further object of some embodiments of the present disclosure is to make efficient use of the time and specialized skills of the radiologists or other medical specialists who are used for reading cases, and to equitably distribute work among those readers.

An object of some embodiments of the present disclosure is to ensure that cases are read by some radiologists (when the cases are radiology cases) before time deadlines that have been assigned for reading them, if necessary even by a radiologist in a different subspecialty, or associated with a different site than the case came from, and similarly for other types of medical imaging cases.

Another object of some embodiments of the present disclosure is to discourage radiologists or other readers from cherry picking cases that are more desirable for the radiologist but less urgent than other cases that are waiting to be read. More generally, an object of some embodiments of the present disclosure is to equitably balance workload, and to ensure that undesirable cases are fairly shared among radiologists or other readers. It should be understood that as used here, “cases that are more desirable for the radiologist” refers to cases that most radiologists would prefer to choose for selfish reasons, for example because they require less work than other cases for the same amount of pay. Similarly, “undesirable cases” refers to cases that most radiologists would prefer to avoid choosing for selfish reasons.

These objects are given only by way of illustrative example, and such objects may be exemplary of one or more embodiments of the invention. Other desirable objectives and advantages inherently achieved by some embodiments may occur or become apparent to those skilled in the art. The invention is defined by the claims as granted.

### Escalation of Unread Cases

An aspect of some embodiments of the invention concerns a computer system that assigns cases involving medical images to workstations of readers, such as medical specialists, for reading, allowing the readers to specify to the computer system their choices of cases to read, but allowing or encouraging each reader to choose from only some cases to the exclusion of other cases. For a case with lower urgency for reading, only a portion of the readers, for example readers who are better qualified or more suitable than other readers to read the case, are allowed or encouraged to choose it for reading initially, but as the urgency for reading the case increases with time, if it is still not read, other readers are added to the portion of readers who are allowed or encouraged to choose it for reading. Allowing or encouraging additional readers to choose a case that has become more urgent is referred to herein as “escalating” the case. If, after escalating a case, it is still not chosen for reading, then optionally the case is further escalated one or more times, adding still more readers to the portion of readers who are allowed or encouraged to choose it for reading.

As used herein, a workstation means any computer with functionality that allows a reader to interact with the computer system in the way required for any of the embodiments of the invention described herein. A workstation need not have special characteristics beyond those of an ordinary personal computer, and need not be used, or usable, for other tasks such as displaying the images that the reader reads.

Having cases exposed to multiple readers, and assigning a case to the workstation of a reader who chooses it, gives readers flexibility in reading the cases they are most suited to read. At the same time, readers do not choose cases from a common list that is shown to all readers, but each reader is shown an individualized worklist of cases to choose from, that takes into account characteristics of the cases and characteristics of the readers, such as matching subspecialties. Escalation of cases, which typically results in exposing them to a greater number of readers if they have not yet been read by a certain time, may help to ensure that cases are read by their deadline. Displaying information about cases on the readers' workstations in a way that indicates to the readers that certain cases have higher priority for reading than other cases, and monitoring readers who too often choose lower priority cases without having a good reason, may help to discourage readers from choosing cases out of order, while giving them the flexibility to do so if they have a good reason.

Optionally, the cases are radiology cases, the cases are assigned to workstations of radiologists, and the criteria used for allowing or encouraging a reader to choose a case may be specific to radiology cases, for example based on radiology subspecialties. Alternatively, the cases are in other medical specialties, for example they are pathology cases, for example involving images of biopsy specimens, assigned to workstations of pathologists for reading. Or the cases involve endoscopy images, and are assigned to workstations of appropriate specialists for the organs that were endoscopically imaged, for example gastroenterologists or a pulmonologists. Or the cases involve dermatology images, and are assigned to workstations dermatologists for reading. Or the cases involve opthamology images, such as retinal scans, and are assigned to workstations of opthamologists for reading. The system and methods have been developed by the inventors for use with radiology cases, and the embodiments of the invention described herein generally refer to radiology cases and radiologists, and give examples from radiology. However, it should be understood that the systems and methods described herein can generally be applied as well to other types of images read by other types of physicians, in some cases requiring appropriate changes. It should also be understood that in some situations and/or in some jurisdictions, the medical imaging cases may be read by other kinds of medical personnel, even if they are not physicians, and references herein to “physicians” are only exemplary.

As used herein, “better qualified to read a case” typically means having a subspecialty that matches the case, for example based on the part of the body that is imaged, or on the modality of imaging. In addition, some radiologists have special expertise at reading cases using a particular imaging modality, such as MRI or ultrasound, and “better qualified to read a case” may also refer to the radiologist being particularly skilled at reading cases that use that modality. In some embodiments of the invention, radiologists are exposed to and/or encouraged to choose a case, optionally for cases that are less urgent for reading, based on other reasons of suitability, for example based on whether the radiologist has an association, affiliation, or working relationship with a radiology site, such as a hospital or clinic, from which the case originated, referred to as the source site or originating site of the case. For each radiologist, there is optionally a radiologist profile that the computer system has access to, which indicates characteristics of the radiologists, such as subspecialties, and associations with sites. The computer system optionally chooses which radiologists to expose a case to, based in part on these characteristics as indicated in the radiologist's profile.

By limiting the pool of eligible radiologists initially, and then expanding the pool to other radiologists when the case is escalated, a tradeoff is optionally made between the conflicting goals of 1) having cases read by radiologists who are best qualified or otherwise most appropriate for reading them, and 2) making sure that cases, especially the most urgent cases, are read as soon as possible, and/or before a deadline. Escalating a case more than once, as it becomes increasingly urgent without being read, may provide a better solution to this tradeoff.

Optionally, the decision whether to expose a given radiologist to a given case, at a given time, and/or whether and to what extent to encourage the radiologist to choose the case, is made automatically by a computer system. Such a computer system is sometimes referred to herein as a “workflow system.” Optionally, the workflow system exposes information about cases to workstations of different radiologists, and/or encourages different radiologists to choose cases, on an individualized basis, depending on data specifying characteristics of the cases and characteristics of the radiologists that is stored in the workflow system. Optionally this is done in a way that balances conflicting requirements, such as the tradeoff described above between having cases read by the best qualified radiologists, and having cases read before their deadlines, or any of the tradeoffs described below. Optionally, the workflow system is configured to at least attempt to balance the conflicting requirements in an optimal way, according to some performance criterion. For example, as described below, an optimal solution to the tradeoff is optionally achieved, or at least approached, by using feedback from measures of performance to adjust parameters that specify which radiologists each case is exposed to as a function of time. It should be understood that having a large number of radiologists working for an organization that provides radiology reading services for multiple medical facilities may make it more difficult for the organization to ensure that cases are read in a timely way, while making efficient use of the radiologists' time and skills, in contrast to a single medical facility that assigns cases only to its own radiologists. On the other hand, if the assignment of cases to radiologists by the large organization is done with the help of such a well-designed workflow system, then the potential exists for making even more efficient use of radiologists' time and skills, than if they were reading cases only for their own medical facility.

Optionally, only some cases are exposed to each radiologist for choosing, and that radiologist is not allowed to choose other cases for reading. As used herein, saying a case is “exposed to” a radiologist means the case is visible to the radiologist, or information about the case is displayed to the radiologist. Optionally, radiologists are only exposed to cases that they are allowed to choose for reading. Optionally, the cases that a radiologist is exposed to, whether they constitute all cases in the system or only some of the cases, are divided into recommended cases, which the radiologist is encouraged to choose, and non-recommended cases, which the radiologist is not encouraged to choose but is allowed to choose. For example, information about recommended cases may be displayed in a bolder and/or darker font than information about non-recommended cases on the radiologist's worklist.

Alternatively, no distinction between “encouraged” and “not encouraged” is made among cases that a radiologist is allowed to choose. In that situation, a radiologist may still be referred to herein as “encouraged” to choose any of those cases, and the set of cases that the radiologist is allowed to choose will be identical to the set of cases that the radiologist is encouraged to choose.

Optionally, the cases that a radiologist is exposed to are completely or partly ranked in order of priority by the workflow system for that radiologist, for example cases closer to a deadline are ranked higher, and radiologists are encouraged to preferentially choose their higher priority cases over their lower priority cases, at least if they do not have a good reason to choose one of their lower priority cases. It should be understood that “priority” here refers to how urgent a case is for medical reasons, and does not refer to how a desirable a case is considered to be by a radiologist for selfish reasons. Optionally, cases are ranked in order of priority within a category, for example trauma cases are ranked in order of priority relative to other trauma cases, but optionally not between categories. Alternatively, cases are ranked in a single order of priority for all categories. Optionally, if a radiologist chooses, or persistently chooses, cases with a lower order of priority over cases with a higher order of priority, this fact is made known by the workflow system to other radiologists, so that peer pressure encourages radiologists to preferentially choose higher priority cases. Peer pressure may also encourage radiologists to choose higher priority cases if measures of performance of a whole group of radiologists, for example radiologists with a particular subspecialty or associated with a particular site, are made known by the workflow system to members of that group, eliciting feelings of group pride or group solidarity. The term “preferred order” may be used herein, with the same meaning as “order of priority,” the order in which the cases are ranked by the workflow system.

Optionally, each case is given a well-defined deadline for reading it, possibly based on a contract with the site that the case came from, and the urgency of a case for reading is based at least in part by how much time remains before the deadline, for example as an absolute time or as a percentage of the initial time to the deadline. Such contractual deadlines are sometimes referred to as Service Level Agreement (SLA) times, and the time interval from when a case is first received, or from when the study is done, until the contractual deadline, may be referred to as the SLA time interval. Alternatively, all or some cases do not have a well-defined deadline, but those cases have an urgency, for example represented by a number, that increases as the case becomes older without having been read yet. Typically cases involving patients who need diagnosis and treatment as soon as possible, such as acute stroke patients or trauma patients, will have a shorter time to deadline, and/or their urgency will start out higher and/or increase faster, than cases involving patients for whom immediate diagnosis and treatment is not so important, for example osteoporosis patients. The increased awareness in recent years of the importance of timely diagnosis and treatment for stroke patients makes it even more important for the workflow system to ensure that cases involving acute stroke patients, at least, are read soon after the study is performed. The order of priority of different cases, at any given time, for a given radiologist, is optionally calculated by the workflow system using a formula or a look-up table for example, from data on characteristics of the cases and characteristics of the radiologists stored in the workflow system, including the time when the study was performed for each case, whether or not the cases have well-defined deadlines.

A case may be escalated at one or more escalation times while it remains unread. Optionally, the escalation times occur at certain fractions of the total time available for reading a case, for example as specified by the SLA. Alternatively or additionally, the escalation times occur at certain absolute time intervals after the time the study was performed. When a case is escalated, for example because it has remained unread for too long, the criteria for exposing the case are changed to be more inclusive, allowing the case to be exposed to more radiologists. For example, initially, a case may be exposed only to radiologists associated with the site of origin of the case, and having a subspecialty that matches the case, as indicated by their profiles. After a first time interval, for example after 40% of the time has elapsed without the case being read, the case is escalated a first time, for example exposing it to radiologists associated with any site, not just the site of origin of the case, but only to radiologists with a subspecialty in that type of case. After a second time interval, for example after 70% of the time interval has elapsed without the case being read, the case is further escalated, exposing it to all available radiologists logged in to the system, even radiologists with different subspecialties. In some embodiments of the invention, there are two, three, or more escalation times, with the criteria for exposing a case becoming more inclusive at each escalation time.

In some embodiments of the invention, the escalation is in principle continuous, though in practice the number of radiologists that a case is exposed to can only increase discretely, by whole numbers. For example, the criteria for exposing a case to a radiologist can depend on a continuous variable listed in a radiologist's profile that characterizes the radiologist's ability or experience, or can even depend on a randomly changing continuous variable assigned to radiologists, and the required value of the continuous variable can be a continuous function of time. In such an embodiment, the number of radiologists that a case is exposed to might increase gradually and smoothly as a function of time, for example increasing by only one radiologist at a time.

In some embodiments of the invention, as noted above, the subspecialty of a radiologist is used as a criterion for exposing cases. Optionally or additionally, the radiologist's expertise in reading cases of a particular modality is used as a criterion for exposing cases. Such expertise is also optionally indicated in a radiologist's profile. It should be understood that whenever the subspecialty of a radiologist is mentioned herein, expertise at reading cases of a particular modality is also optionally included, even when this is not explicitly stated.

Optionally, the times at which cases are escalated, and even the number of times at which cases are escalated, are different for different categories of cases, for example it is different for more urgent categories of cases, such as acute stroke cases or trauma cases, than for other cases.

Optionally, cases in each of different categories, for example categories with different levels of urgency, are displayed to the radiologist grouped together in different sub-worklists.

Optionally, the escalation times depend on the work shift, for example as specified by the radiologist when logging in, or depend on the time of day or day of the week even within a given work shift, which may be advantageous since the number of radiologists who are working may depend on the shift, as well as on the time of day and the day of the week within a given shift. The rate at which new cases come in will generally depend on the time of day and the day of the week, and may be better matched to the number of radiologists working at some times of day, and some days of the week. Also, how quickly radiologists work on average may depend on work shift, or on time of day even within a work shift. At times of day and days of the week when there are enough radiologists available to easily read the cases coming in by their deadlines, it is potentially advantageous to set the first escalation time relatively late, so that more radiologists can read cases for their own sites, to maintain their relationships with their sites. It is also potentially advantageous, at those times, to set the second escalation time relatively late, so that more cases are read by radiologists with matching subspecialties. At times of day and days of the week where the number of available radiologists is only marginally adequate for the number of cases coming in, it is potentially advantageous to set the escalation times relatively early, to ensure that cases are read by their deadlines. The times of day and days of the week where these conditions occur may depend on local circumstances, and the rules for the escalation times can be set accordingly.

Optionally, the rules for when cases are escalated, and/or the criteria for exposure at each escalation time, are adjusted manually by administrators, and/or dynamically adjusted automatically by the system, in response to one or more measures of efficiency of the workflow, for example one or more of the fraction of cases that are read by the SLA, the fraction of cases that are read by a radiologist whose subspecialty matches the case, and the fraction of cases read by a radiologist associated with the site of origin of the case. Such automatic or manual adjustments are also optionally made in response to the current number of unread cases of different types, or the rate at which new cases of different types are coming in. Optionally, these adjustments are also made in response to how many radiologists associated with the site of origin of a case, and/or with a subspecialty that matches the case, are logged in. For example, cases are optionally escalated earlier, or even from the beginning, exposing them to radiologists associated with different sites and/or having non-matching subspecialties, whenever there are fewer radiologists, or no radiologists, logged in who are associated with the site of origin of the case, and/or who have a subspecialty matching the case. In such circumstances the case is less likely to be read before it is escalated. These adjustments in the escalation time, and/or in the rules for which radiologists are exposed to which cases before and after each escalation time, whether done manually or automatically, can potentially result in a better tradeoff between having cases read sooner, and having cases read by radiologists with a subspecialty that matches the case, and who are associated with the site of origin of the case.

### Displaying Escalated and Non-Escalated Cases on the Same Worklist

An aspect of some embodiments of the invention concerns allowing or encouraging only a portion of radiologists to choose a case initially, and escalating the case, allowing or encouraging more radiologists to read it, as it becomes more urgent without being read. Information about the cases that a given radiologist was allowed or encouraged to read initially, and information about the cases that the radiologist was allowed or encouraged to read only when they were escalated, are displayed in a single worklist, for example displayed contiguously on the radiologist's workstation screen, optionally listed in an order of priority, for example as a linear list with cases listed one below the other. Displaying information about these cases together in a single worklist may make it easier for the radiologist to immediately see when new cases, often higher priority cases that have just been escalated, have appeared on the worklist, while still allowing the radiologist to keep in mind what other cases are available, without the need to switch back and forth between different lists.

In some embodiments of the invention, information about all cases that a radiologist is allowed to choose is displayed in a single worklist, but with different categories of cases listed separately in different sub-worklists. Optionally, each sub-worklist is displayed as a single linear list of cases in order of priority. Optionally, the different sub-worklists are displayed sequentially one above another, for example in order of level of urgency, for example with a sub-worklist for acute stroke cases at the top, and a sub-worklist for outpatient cases at the bottom. Alternatively, the different sub-worklists are displayed side by side. Optionally, the entire worklist, even if it is divided into sub-worklists, is displayed in a single user interface element, such as a single window, that can be moved as unit around on the screen. Optionally, in this situation, information about all cases is visible to a user at the same time. Alternatively, information about all cases is not visible to the user at the same time, but the user can scroll up and down to see the cases listed in different sub-worklists, and/or the user can expand each sub-worklist, for example by clicking on it. Optionally, instead of displaying the entire worklist in a single user interface element, two or more different sub-worklists are displayed in different user interface elements, such as different windows, that can be moved around the screen independently. Even if different sub-worklists are displayed in different user interface elements, optionally the cases that the radiologist was allowed or encouraged to read initially, and the cases that the radiologist was allowed or encouraged to read only when they were escalated, if they are in the same category, are displayed together in the same sub-worklist, for example displayed contiguously on the radiologist's workstation screen, optionally listed in an order of priority, for example as a linear list with cases listed one below the other. Optionally, the entire sub-worklist is displayed in a single user interface element, such as a single window, that can be moved around on the screen. Optionally, information about all cases in that sub-worklist is visible to a user at the same time, or alternatively, even if information about all cases in that sub-worklist is not visible at the same time, the user can scroll up or down to see different cases, and/or the user can expand a group of cases, for example by clicking on it or by using another user interface element, to see a list of all the cases in the group. Displaying the entire worklist in one window has the potential advantage of being easier for the user to pay attention to other sub-worklists while looking at one sub-worklist. Displaying different sub-worklists in different windows has the potential advantage of having room to display more cases.

Discouraging Radiologists from Choosing Lower Priority Cases (“Cherry-Picking”)

An aspect of some embodiments of the invention concerns discouraging radiologists from choosing cases with too low a priority, too often, over higher priority cases, at least if they do not have good reasons for choosing lower priority cases. Radiologists who choose lower priority cases over higher priority cases without good reasons are said to “cherry pick;” this practice may interfere with efficient workflow, and may be unfair to their colleagues. Optionally, the workflow system keeps track of which radiologists choose lower priority cases too often, and this information is available to administrators. Optionally, the workflow system has a mechanism for radiologists to provide reasons if they choose lower priority cases, and optionally they are not discouraged from choosing cases with too low priority, if they provide good reasons for choosing them. Good reasons that a radiologist might provide to the workflow system for choosing a lower priority case optionally include 1) the radiologist does not feel qualified to read any of the higher priority cases available for choosing, and 2) the radiologist believes, for clinical reasons, that the lower priority case should be considered more urgent than the available higher priority cases. A radiologist who cannot provide the workflow system with a good reason might in fact be motivated to choose a lower priority case because the radiologist expects it to require less work for the amount of money it pays, than a higher priority case. For example, reading a CT of the spine may always pay the same amount of money, but if the patient has a history of multiple prior surgical procedures, the CT would likely take substantially more time to read than if the patient is a healthy young adult with mild back pain.

By allowing radiologists to choose some cases that are considered lower priority than other cases waiting to be read, but discouraging them from doing so if they do not have good reasons, a tradeoff may be made between the conflicting goals of 1) giving radiologists freedom of choice, in order to benefit from their clinical insights that some cases are more urgent, and 2) preventing radiologists from cherry picking more desirable but less urgent cases, in order to be fair to other radiologists, and in order to ensure that more urgent cases are read as soon as possible, and/or before a deadline. Another, related tradeoff is between the goals of giving radiologists freedom of choice, and reducing the need for radiologists to spend time deciding which cases to choose.

Additionally or alternatively, sanctions are instituted for radiologists who too many times choose cases with too low priority, at least if they do not have, or do not provide, a good enough reason for their choices. A design goal is to discourage radiologists who might want to “cherry pick” a lower priority case that they consider more desirable, because it is expected to involve less work, and/or is better reimbursed, rather than a higher priority case that they consider undesirable, because it is expected to involve more work and/or is not as well reimbursed. Examples of cases that are expected to involve more work are cases involving older patients, patients who have multiple prior comparison studies, or patients with prior surgery, vascular cases, and cases involving inpatients or ICU patients. A cherry picking radiologist may avoid choosing such cases. The instituted sanctions may deter radiologists from engaging in such cherry picking, even if the sanctions never have to be used.

Optionally, if the workflow system decides, based on monitoring a radiologist's choices of low priority cases, that the radiologist is engaged in cherry-picking, then the cherry-picking radiologist's behavior is made known to other radiologists. Knowing in advance that one's cherry-picking will be known to other radiologists, can deter cherry-picking by social pressure.

Optionally, even if the workflow system does not identify individual cherry-pickers, to the system provides information to other radiologists that a particular group of radiologists, for example from one subspecialty and from one site, have not been keeping up with their highest priority cases. Such information is provided, for example, because cases are escalated when they get close to their deadlines, and they may eventually appear as the highest priority cases on the worklists of all available radiologists. Optionally, cases on worklists are identified by site of origin, and by subspecialty, and if an unusually large number of cases from one subspecialty and/or one site of origin starts to appear near the top of the worklists of radiologists with other subspecialties and/or not associated with that site, it will be apparent to all the available radiologists that radiologists from this particular subspecialty and/or site are falling behind in reading their highest priority cases, possibly due to cherry-picking. This prospect can motivate members of that group not to engage in cherry-picking, out of group pride or solidarity, to improve the performance of the group.

Optionally, the workflow system has a mechanism by which sanctions are imposed by administrators. Additionally or alternatively, the workflow system has a mechanism for imposing sanctions automatically. Examples of sanctions that might be imposed include assigning cases that most radiologists would find undesirable, by only allowing the radiologist to choose such cases, for a period of time. Optionally, when cases are received, their characteristics are analyzed to judge whether they are likely to be considered desirable or undesirable cases by most radiologists, for example for the reasons listed above, and this information is included as part of the supplementary information associated with each case.

Optionally, cases that a given radiologist is allowed to choose from are designated by one of only two degrees of priority for that radiologist, for example “recommended” and “not recommended,” and the sanctions are optionally instituted for radiologists who too often choose cases that are not recommended, for example more than a predefined number of times within a predefined time interval. Designating a case as “recommended” to a radiologist is referred to herein as “virtual assignment” of the case to that radiologist. Optionally, a fixed number of cases, for example the 2, 3, or 4 cases that are considered to be of the highest importance for that radiologist, are recommended, and the other cases are not recommended. Optionally, a fixed number of cases are recommended in each of several categories, for example acute stroke, trauma, in-patient, etc., and the other cases are not recommended. The fixed number need not be the same for all categories. Optionally, the fixed number is dynamically adjusted, manually by an administrator or automatically by the system, in response to measures of performance, in an attempt to improve the overall performance of the system according to some measure.

Alternatively, the cases are designated by one of more than two levels of priority, and/or at least some of the cases are ranked by order of priority, with the rankings or levels of priority optionally only specified relative to other cases within a category, such as acute stroke cases or trauma cases. In this circumstance, the workflow system optionally keeps track of radiologists whose choices in on average have level or order of priority below some threshold, and/or who too often choose cases with level or order of priority below some threshold.

In some embodiments of the invention, cases are ranked in order of priority only within each of several categories of cases, each category optionally displayed as a separate sub-worklist, for example acute stroke cases, trauma cases, ER cases, in-patient cases, etc., but there is no ranking of order of priority between categories. In this situation, most radiologists may understand that they should read cases from the most urgent categories first, for example acute stroke cases and trauma cases, and only read cases from less urgent categories if there are no cases waiting to be read in the more urgent categories. In other embodiments of the invention, all cases displayed to a given radiologist are ranked in a single order of priority, and optionally information about them is displayed in a single merged worklist, including cases in different categories. Some examples of how such a single order of priority can be formulated are described below under the heading, “Setting an overall order of priority for cases from different categories.” A potential advantage of such an arrangement is that radiologists may be less likely to completely neglect cases in categories of low urgency, such as out-patient cases, since some of the out-patient cases can be given higher priority when there are not too many acute stroke and trauma cases that are unread close to their deadline. An alternative or additional method of steering radiologists to choose cases in neglected categories, while still listing the different categories of cases in different sub-worklists, is described below in the section with the heading, “Dynamically adjusting exposure parameters.”

Optionally, a case is escalated, meaning that additional radiologists are allowed and/or encouraged to choose the case, when the urgency for reading the case has increased, because more time has passed without the case being read. Any of the options described above, for escalating a case, may be used.

### Individualized Worklists

A broad aspect of some embodiments of the invention concerns displaying to each radiologist a worklist of cases that the radiologist is allowed to choose from for reading, optionally listed in an order of priority. A computerized system generates the worklists, including the order of priority within each worklist, taking into account characteristics of the cases, such as type of study and site of origin, and characteristics of the radiologists, such as a subspecialty, a relationship with a site, how much work they have done that month, and a recent history of cherry-picking. Due to individual differences between radiologists, even radiologists with the same subspecialty and working for the same site, the worklists will generally be individualized, and could list a different set of cases, and/or a different order of priority of the cases, for each radiologist, though in a given instance two or more radiologists may happen to have the same set of cases listed on their worklists in the same order of priority. In general, each case is included on the worklist of a plurality of radiologists, and the case is read by the first radiologist who chooses it for reading. However, it might happen, in a given instance, that a case appears on the worklist of only one radiologist, for example if only one radiologist from a particular site with a particular subspecialty is logged in at that time.

### Displaying Statistics on Unread Cases

An aspect of some embodiments of the invention concerns a user interface for displaying worklists for radiologists, which also displays the number of unread cases in the system, in different categories. Such a display may facilitate selection of cases by radiologists, by displaying implicit and explicit information in real time relating to workflow, and any bottlenecks that may arise. For example, the number of unread cases in each subspecialty is displayed, and/or the number of unread cases coming from each site of origin. An unusually large and/or rapidly increasing number of unread cases from one site, for example, may indicate a bottleneck in reading for that site. Optionally, a small table, sometimes referred to as a “dashboard,” is additionally displayed, showing the number of unread cases in each subspecialty from each site of origin. Additionally or alternatively, this information is presented in a graphical element, such as a bar graph, a color-coded element or a finable shape. By looking at these numbers, as well as by seeing how they change over time, any user of the system, whether a radiologist or an administrator, can tell at a glance if some group of radiologists, for example radiologists of one subspecialty, or radiologists working for one site, is falling behind in their work, and the user may take measures to try to solve the problem. For example, a member of a group that is falling behind might try to work faster, or stay at work longer. An administrator might try to call in extra radiologists in that subspecialty, who are not currently logged in but who are on call for emergencies, possibly with overtime pay. There may also be measures that the workflow system can take automatically to try to solve the problem. For example, the workflow system could automatically send an email or make a pre-recorded phone call to radiologists in that subspecialty who are not currently logged in, urging them to log in.

### Dynamically Adjusting Exposure Parameters

In some embodiments of the invention, criteria that determine which cases are exposed to which radiologists at a given time, or how some cases are given preference over other cases for different radiologists at a given time, are adjusted dynamically, manually by an administrator or automatically by the system. The adjustment is made in response to one or more measures of performance which provide feedback on the effect of the adjustment in the criteria, in order to try to improve the overall performance of the system, by some measure. Optionally, the measures of performance are monitored continuously, or at frequent intervals, to facilitate this feedback process. The adjustment in criteria may be accomplished by changing the values of parameters that define the criteria. Whether the criteria are changed manually or automatically, it is potentially advantageous to use a relatively small number of measures of performance, and a relatively small number of parameters, with the measures of performance and the parameters judiciously selected to make it apparent and intuitive which parameters should be changed and in what direction, in order to change the measures of performance in a given direction, and in order to improve overall performance. If the parameters are changed automatically in response to the measures of performances, then optionally the software that controls the change in parameters uses a feedback mechanism, such as any standard feedback method known from control theory, for example in order to maximize a measure of overall performance, or in order to bring the different measures of performance closer to desired values. The feedback method is designed, for example, to provide a stable response to changes in the parameters, for example by including a damping term to avoid overshooting. The measure of performance is optionally calculated as a running average. For example, the running average includes all cases that were received within a specified time period before the measure of performance is calculated, or includes all cases that were waiting to be read at a specified time before the measure of performance is calculated, and updated values of the measure of performance are repeatedly calculated, for example periodically at specified times. For purposes of achieving stable feedback, it is potentially advantageous if the sets of cases used for successive calculations of the measure of performance have considerable overlap, for example at least 50% or at least 80% or at least 90% overlap, so that the measure of performance changes relatively smoothly over time.

If some of the cases exposed to a radiologist are given preference over other cases, then this is optionally indicated to the radiologist in the way that information about the cases are displayed. For example, the cases are listed in an order of priority, with cases of higher priority higher up on a list than cases with lower priority, and by this display the radiologist is encouraged to preferentially choose the higher priority cases over the lower priority cases. Alternatively or additionally, the cases that are exposed to a radiologist are divided into recommended and non-recommended cases, for example by displaying information about the recommended cases in a darker or bolder font, and/or displaying information about the non-recommended cases in a lighter colored font, for example “grayed out,” and the radiologists are encouraged to choose only the recommended cases, unless there is a good reason to choose a non-recommended case. Alternatively, no cases, among the cases that are exposed to a radiologist, are given preference over other cases for which information is displayed, and in this case the radiologist can be considered “encouraged” to choose any of the cases.

As an example of criteria that define which cases are exposed to which radiologists at a given time, the criteria specify that cases are escalated, exposing them to an additional group of radiologists, after a fraction P of the time between when the study was done, or when the case was received, and the deadline (SLA) for reading the case. In some embodiments of the invention, instead of a single parameter P, there are multiple parameters P1, P2, . . . , corresponding to more than one escalation time for each case. The value of P or of the multiple parameters P1, P2, . . . can be adjusted dynamically, based on measures of performance, in order to improve performance. For example, on a day when there are fewer than the usual number of radiologists logged in, better performance may be achieved by using a lower value of P, so cases are escalated earlier.

Optionally, instead of or in addition to changing a parameter such as P directly, in response to the measures of performance, a rule for how P varies in time is changed, in response to measures of performance. For example, if it is noticed that, over several days, there are consistently more radiologists logged in, or the radiologists work more quickly, in the morning than in the afternoon, causing an increase in unread cases in the afternoon, then a rule for how P changes over time is changed, making P lower in the afternoon than in the morning every day, possibly with the change in P occurring slightly in advance of the anticipated change in the number of unread cases.

Measures of performance may include, for example, one or more of the fraction of cases not read by their deadline, for different categories of cases, the fraction of cases not read until close to their deadline by some definition, the fraction of cases read by radiologists with a subspecialty that does not match the case, and the fraction of cases read by radiologists not associated with the site of origin of the case. If changing the parameter P is used to adjust the criteria, and if more than a threshold fraction of cases during a set time interval and/or out of a set number of cases are not being read by their deadline or close to their deadline, then optionally P is lowered to improve performance. But if more than a threshold fraction of cases during a set time interval and/or out of a set number of cases are not being read by matching subspecialists or radiologists associated with the same site, and all or almost all cases are being read well before their deadline, then optionally P is raised, to improve performance. Optionally, how much P is raised or lowered depends on how much the threshold is exceeded.

As another example of criteria that can be adjusted dynamically, in some embodiments of the invention, a reader is encouraged to choose only a limited number of cases N, at least among one category of cases, for example among acute stroke cases, or among trauma cases. For example, the cases that a reader is allowed to choose are ranked in order of priority, and the reader is encouraged to choose only one of the top N cases in order of priority, which are recommended, as shown for example by displaying information about them in bold font, and the reader is not encouraged to choose lower priority cases, which are not recommended, as shown for example by graying them out in the display. If N is the number of cases that a reader is encouraged to choose in one category, then N need not be the same for all categories. Alternatively, the lower priority cases are not listed at all, and the reader is not allowed to choose them. Optionally, particularly in that circumstance, no subset of the listed cases is given preference over the other listed cases, and the reader may be considered “encouraged” to choose any of them. Optionally, the number N is adjusted dynamically, in order to improve performance. For example, if more than a threshold fraction of cases are being chosen that are lower than the highest priority or lower by more than a threshold amount, even though they are recommended, and the number of cases with priority above a threshold are accumulating unread at greater than a threshold rate, then N is optionally lowered. If the readers as a whole are keeping up with the highest priority cases, as indicated for example by the number of such cases not having increased over a specified time interval, then N is optionally raised, for example to give the readers more flexibility, and possibly to increase the number of cases read by readers with a matching subspecialty, and/or by readers associated with the same site. Optionally, instead of or in addition to directly changing N, a rule for how N varies over time, for example how it changes in different escalation periods, is changed.

When different categories of cases (such as acute stroke, trauma, in-patient, etc.) are listed on different sub-worklists, each with its own order of priority, then it sometimes happens that one or more sub-worklists are neglected, often a sub-worklist for a category of lower urgency. In such a situation, the values of N for different sub-worklists are optionally adjusted differently, to steer more readers to choose cases in a sub-worklist that is being neglected. For example, a running average of the turnaround time for reading cases (optionally with outliers excluded) is calculated for each sub-worklist, and the ratio of average turnaround time to SLA, for each sub-worklist, is used as a measure of performance. If this measure of performance is well below 1, by an adjustable threshold, for a first sub-worklist, and there are cases that have not been read by the deadline, or close to the deadline, on a second sub-worklist, then optionally N is temporarily reduced, for example for several minutes, possibly to zero, for the first sub-worklist, encouraging or forcing readers to choose cases from the second sub-worklist instead of from the first work sub-list. Optionally, when the measure of performance falls to a value below 1 by a threshold for the second sub-worklist, or if more than a threshold number of unread cases get close to the deadline for the first sub-worklist, then N is increased again for the first sub-worklist. In some embodiments of the invention, instead of or in addition to using average turnaround time as a measure of performance for comparing workflow in different categories, one or more other measures of performance is used, for example the fraction of cases that are being read by their deadline, or the fraction of cases that are being read by a specified time interval before or after their deadline, for example the fraction of cases that are being read by 80% of the SLA time interval, or by 50% of the SLA time interval, or by 110% of the SLA time interval.

In some embodiments of the invention, as described below in the section headed “Setting an overall order of priority for cases from different categories,” information about cases in different categories is displayed with a single order of priority, for example as a merged worklist that combines cases from different categories, and the order of priority is determined by an index of importance that depends on both the time to deadline and the category. If cases from one category are being neglected, as indicated by the measures of performance described above, then optionally parameters that determine the index of importance for cases in different categories are changed adaptively, in response to the measures of performance. For example, cases in the neglected category are given a higher index of importance, and/or cases in other categories are given a lower index of importance.

Optionally, any or all of the measures of performance that are used to adjust the criteria are displayed to the radiologists, for example on their worklists, or on a specific sub-worklist that the measure of performance applies to. This has the potential advantage that the radiologists will understand why they are being limited in which cases they are allowed or encouraged to choose, possibly in ways that will make them choose cases that they would prefer not to choose. This understanding may make the radiologists less resentful about this, and perhaps give rise to a feeling of group solidarity, and a willingness to make sacrifices to improve overall performance.

Optionally, the adjustment in the criteria is made manually by an administrator, using human judgment in deciding how to change the parameters, for example after looking at the measures of performance. Alternatively, the adjustment in the criteria is made by a fixed algorithm, which determines how the parameters are changed in response to the measures of performance which provide feedback on the effect of the changes in parameters, and no human judgment is involved. Optionally, the fixed algorithm involves one or more of a set of rules, a look-up table, discrete and continuous formulas, a neural network, and machine learning. The fixed algorithm optionally predicts how the measures of performance will change in response to changes in the parameters, based for example on past experience, and uses these predictions to find new values of the parameters that are expected to optimize or at least improve performance.

Setting an Overall Order of Priority for Cases from Different Categories

An aspect of some embodiments of the invention concerns a method of setting an overall order of priority for cases, for a given radiologist, from all categories, or from two or more categories, corresponding to different levels of urgency, such as acute stroke cases, trauma cases, in-patient cases, etc. Such an overall order of priority is used, for example, when cases in different categories are listed together in a single merged worklist, instead of listing cases in separate sub-worklists, one for each category, with the order of priority of cases within each sub-worklist based, for example, on the time to deadline. Although it is also possible to simply list cases from different categories in order of time to deadline, in practice this may not very well reflect the true order of importance for reading the cases, for that radiologist. For example, an out-patient case that is 5 minutes past its deadline would show up as higher priority than an acute stroke case that is 1 minute past its deadline, but for medical considerations, the stroke case should have a higher priority for reading. On the other hand, if all cases in a category of higher level of urgency, such as ICU cases, are listed higher in priority than any cases in a category of lower level of urgency, such as out-patient cases, then the lower level cases may never be read at all, or may only be read long past their deadlines, and this is also not a desirable situation.

The ability to overcome these problems, in some embodiments where an overall order of priority is found for cases in different categories, is a potential advantage of basing this order of priority on an index of importance, which is calculated for each case from an index of importance function, which depends on time, for example on time to deadline or on elapsed time since the study was done, and on characteristics of the case. The index of importance is generally higher for a case with a shorter time to deadline, than for a case with a longer time to deadline and otherwise identical characteristics, including being in the same category of level of urgency; however, in some embodiments of the invention the index of importance is calculated from the time to deadline by binning, with cases having times to deadline within a same range having the same index of importance. The index of importance is also generally higher for a case in a category of higher level of urgency, than for a case in a category of lower level of urgency, having the same time to deadline and otherwise identical characteristics. Optionally, the index of importance for a case also depends on other characteristics of the case, for example the site of origin or the identity of the attending physician. It should be understood that the index of importance is used to set an order of priority of cases for the worklist of a given radiologist, and the index of importance of a given case may be different for different radiologists. For example, the index of importance may depend on whether the radiologist has a subspecialty that matches the case.

The order of priority for different cases at a given time is generally the order of the index of importance for the cases. However, if there are two or more cases with the same index of importance, which is likely to happen if the index of importance is found by binning the time to deadline, then the order of priority among those cases optionally depends on the time to deadline, with cases with shorter time to deadline having higher priority. Alternatively, the order of priority for those cases depends on whether the radiologist has a matching subspeciality, or is associated with the site of origin of the case, or the order of priority may be arbitrary, for example based on an ID number of the patient, or may be random.

There are several ways to make a tradeoff is made between time to deadline and category, and possibly other characteristics of the case, in defining the index of importance function. In an exemplary embodiment of the invention, the index of importance function is the sum of a component that depends only on time to deadline, in a monotonic but nonlinear way, and a component that depends only on the category. The component that depends on time to deadline has a finite range of values, going from zero at large positive time to deadline (the largest SLA time interval for any category), to a maximum value at large negative time to deadline, i.e. long after the deadline has passed. Within a range around zero time to deadline, for example between +15 minutes and −15 minutes, the component increases linearly, or at least approximately linearly, as the time to deadline decreases. For larger positive or negative values of the time to deadline, the component varies more slowly with time to deadline, approaching its maximum value for large negative values. It should be kept in mind that for higher urgency categories, this component generally does not start at zero, if the time to deadline initially is a smaller positive number than the largest SLA time interval for any category, and for cases in these higher urgency categories, the total range between maximum and minimum value of this component is smaller.

In some embodiments of the invention, the index of importance function is a different function of time to deadline for different categories, not just differing by a constant, but having a different shape. In those embodiments, the range between maximum and minimum value for cases in a given category need not be smaller for categories of higher level of urgency.

The behavior of the index of importance depends on whether, and to what extent, the range between the maximum and minimum value that the index of importance function takes on, at different times to deadline, for a given category, overlaps the range for other categories. In an exemplary embodiment of the invention, the total range between maximum and minimum value of the index of importance function for acute stroke cases, the highest level of urgency, does not overlap, or barely overlaps, the total range for the category of the second highest level of urgency (trauma cases), and does not overlap the range for other categories. This means that acute stroke cases will always have a higher index of importance than any other cases, or at most, a trauma case that is well past its deadline might have a slightly higher level of importance than an acute stroke case that is well before its deadline. This result accords with the intuition that acute stroke cases should always or almost always take priority over any other cases. For the lower levels of urgency, including trauma cases, and especially for in-patient cases and out-patient cases, there is optionally overlap in the ranges between the maximum and minimum value that the index of importance function takes on. For these levels of urgency, a case with lower level of urgency that is close to or past its deadline might very well have a higher index of importance than a case with higher level urgency that is well before its deadline, which is also in accord with intuition, and may be useful in ensuring that cases at the lower levels of urgency are not neglected.

In some embodiments of the invention, an index of importance is used for determining an order of priority for reading cases even in a single category displayed in a single sub-worklist, instead of basing the order of priority only on the time to deadline. This is potentially useful in several situations, as shown in the following examples. First, even for cases within a category, the index of importance function may depend on other characteristics of the case, such as site of origin, and this can be used to give some preference to reading cases from one site of origin over another site of origin, even within a category. Second, if the index of importance function is a binned monotonic function of the time to deadline, then cases within the same bin need not be read in order of time to deadline, but other criteria can be used, for example giving preference to cases with a subspecialty that matches the radiologist. It may be advantageous to use differently sized bins for different categories of cases. For example, it may be advantageous to use narrower bins for cases that are especially time critical, such as acute stroke cases, and wider bins for cases, such as in-patient or out-patient cases, that are not so time critical, and for which it is relatively more important for the case to be read by a matching subspecialist. Finally, for some categories of cases, the index of importance is optionally not a monotonic function of time to deadline at all. For example, if an acute stroke case fails to be read within the window of opportunity for saving tissue that has had its blood flow cut off due to an ischemic stroke, then the importance of reading the case may decrease, compared to other acute stroke cases that are still within the window of opportunity, and this is optionally reflected by using an index of importance function for acute stroke cases that is not a monotonic function of time of deadline, but first increases and then decreases with decreasing time to deadline, or with increasingly negative time to deadline. This is optionally also done for any category of cases for which there is a critical time for treatment that has passed.

A non-monotonic index of importance function may also be useful for systems in which there is no expectation that all cases will eventually be read. For example, it may be known that the number of working radiologists is not adequate for eventually reading all the cases received by the system, and, depending on the number of radiologists logged in at a given time, and on the number of cases being received at a given time, it is desired to eventually remove some of the less urgent cases from the system without reading them at all, but not to do this with more cases than necessary. In such a situation, for lower urgency categories of the cases, the index of importance function optionally starts to decrease with increasing time past the deadline, until, at a specified time past the deadline, the index of importance function reaches a low value such as zero, and the case is then permanently removed at least from all regular worklists. Optionally, the case is archived on a special worklist which radiologists can get access to. By having the index of importance of the case gradually decrease over a period of time, rather than removing it abruptly from all worklists when its index of importance is still high, the possibility remains that the case will still be read, if the number of logged in radiologists increases more than expected or the number of new cases of relatively high urgency is lower than expected. But the case will have lower priority than cases that are more recent, reflecting the fact that, as time goes on, it becomes less and less important to read it at all. Images from cases that are not read by a radiologist as part of the workflow system may still be viewed by the attending physician, or by another physician or medical worker who is caring for the patient, and they may draw their own medical conclusions from the images without the benefit of a radiologist's report.

In the foregoing description of using an index of importance to set the order of priority of cases, the index of importance function and the order of priority have been described as depending on the time to deadline. However, since the time of the deadline generally depends on when the study was done or when the case was received, and on characteristics of the case such as its category, this entire description of using the index of importance can be expressed instead in terms of time since the study was done, or time since the case was received, rather than in terms of time to deadline. In fact, an index of importance can be used to set the order of priority of cases, for cases in different categories or for cases in a single category, even if deadlines for cases are not defined at all, by expressing the index of importance function as a function of the time since the study was done, or the time since the case was received.

Exemplary algorithms for calculating the index of importance are described below in the section headed “Exemplary algorithms for index of importance.”

### Using Imaging Analytics to Help Determine Urgency of a Case

An aspect of some embodiments of the invention concerns the use of imaging analytics to help determine the urgency for reading a case, and in particular to determine when a case requires immediate treatment more urgently than other cases that cannot be easily distinguished from it by clinical symptoms alone. This use of imaging analytics may be considered a triaging function. One or more images in each received imaging case are analyzed by imaging analytics circuitry, for example a computer running image processing software, to detect the presence or absence of one or more features that would indicate that the case has a high degree of urgency for reading. An example of such a feature is free air in the abdomen, air that is not within the bowel, which, for a patient who has not recently had abdominal surgery, generally indicates a perforated bowel which requires immediate treatment. Another example concerns screening a large number of out-patients from a population of apparently healthy people for a medical condition, for example a disease condition, that is found in only a small minority of the population being screened, but that could require treatment for any people who have the condition. Imaging analytics could look for a feature that indicates that a patient has the condition, or has a good chance of having the condition, efficiently picking out those cases from the cases of the great majority of normal patients. Reading those cases can then be given a higher urgency than reading the other cases.

When a feature indicating a medical condition is detected, the increased urgency is optionally taken into account in the criteria used for determining which readers are allowed to choose the case for reading, and which readers are encouraged to choose the case for reading, as a function of time. For example, the case is exposed to more readers, optionally including readers not associated with the site of origin of the case, and readers with subspecialties that do not match the case. Optionally, this is done immediately, i.e. the case is escalated from the beginning. Alternatively, the case is not escalated immediately, but is escalated sooner than if the feature were absent. Optionally, the case is displayed to readers with a higher order of priority than if the feature were absent, optionally with a higher order of priority than other cases with closer reading deadlines. If the order of priority of different cases is set by an index of importance as described above, then optionally the higher priority is implemented by giving the case a higher index of importance. Optionally, the reading deadline for the case is set earlier than if the feature were absent.

### Goals of Software Design

A possible goal for designing the software for some embodiments of the workflow system, and setting the values of its control parameters, is to optimize the tradeoffs described above under the headings “Escalation of unread cases,” and “Discouraging radiologists from choosing low priority cases.” This is done, for example, by maximizing some quantitative measure of workflow efficiency, but it should be understood that a particular configuration of the software may not succeed in reaching such an optimum. Alternatively, the software is designed and the control parameters are set not necessarily with any expectation of optimizing performance with respect to the tradeoff, but only to obtain a reasonable compromise that takes the tradeoffs into account.

The quantitative measure of workflow efficiency is based, for example, on some combination, for example a linear combination, of several different measures of performance, including any of those described in the section headed “Measures of performance.” Examples of control parameters whose values can be adjusted to maximize or at least increase the workflow efficiency, and exemplary methods of adjusting the values, are given in the sections headed “Dynamically adjusting exposure parameters” and “Using feedback to control exposure parameters.”

Recent trends in medical technology may make some of these tradeoffs even more acute. For example, the increasing complexity of the information in medical images may make it more important to have the images read by radiologists with special training to read them. At the same time, radiologists who specialize in reading new types of medical images may have less skill and/or interest in reading conventional x-ray images, which still make up about half of all radiology images. These trends are potentially important to take into account in designing a workflow system for assigning cases to radiologists. In particular, these trends may make it even more important for such a system to take into account the number and types of cases waiting to be read, and the number and characteristics, including subspecialties, of radiologists currently available for reading cases.

### Measures of Performance

Optionally, the software provides administrators with analytic measures of its performance, optionally in real time, which can be used to tune it over time by changing values of the control parameters, and/or by reconfiguring the software in other ways, to try to find what values of the control parameters and what software configuration work best. Control parameters may include, for example, rules that govern which radiologists will be exposed to which cases, and in what order of priority different cases will be listed for different radiologists, when cases become more urgent as more time elapses without anyone reading them. Optionally, the measures of performance of the radiologists as a whole are also available to the radiologists, and/or the measures of performance of a particular radiologist are available to that radiologist, which may help radiologists adjust their behavior, and their mutual cooperation, to achieve better performance.

One measure of performance is the percentage of cases that are read within an acceptable time, for example within the deadline determined by SLA contracts. The percentage of cases that are read within a specified fraction of the SLA time interval, for example within 80% of the SLA time interval, or within 50% of the SLA time interval, is also a useful measure. If this percentage is lower than normal, or is getting lower, that might indicate that there is a serious risk that soon some cases will remain unread at the deadline, even if that has not happened yet, and that there is a need to take measures to prevent that from happening, such as making adjustments in parameters of the workflow system, or finding more radiologists to read cases. In circumstances where a relatively large number of cases are not being read by the deadline, it may also be useful to use the percentage of cases that are not read by a specified time interval after the deadline, for example by 110% of the SLA time interval, for example as a trigger to initiate steps to remediate the situation. These measures of performance are potentially especially useful if they are broken down by categories of level of urgency, or by the subspecialty of the case, or by the site of origin of the case, since the measures of performance, broken down in that way, may show what measures should be taken to improve performance. Even without making changes in parameters of the workflow system, or obtaining additional radiologists, displaying these measures of performance to the radiologists may help to improve performance, for example by showing that one group of radiologists is falling behind and encouraging them to work faster, or by showing radiologists that cases in one category of level of urgency are not being read quickly enough, and encouraging radiologists to choose those cases. Furthermore, if the criteria for exposing cases to radiologists are adjusted, limiting the cases that radiologists are allowed or encouraged to choose, in response to these measures of performance showing that one or more categories of cases are not being read soon enough, then displaying these measures of performance to the radiologists, so that they understand why they are being limited, may make them feel less resentful and increase their feelings of solidarity with other radiologists.

Another measure of performance is the workflow efficiency, expressed as a weighted or unweighted average over the different radiologists, for example as a mean or a median, with the workflow efficiency of a given radiologist measured by cases read per unit time, optionally weighted according to the difficult of reading the case, or by the number of Relative Value Units (RVUs, a standardized metric reflecting the physician work effort required to interpret a typical case) or other accounting units, earned per unit time.

Another measure of performance is the fraction of cases, or the fraction of radiologists' time spent reading cases, that match the area of specialization of the radiologist reading them. Ideally, this should be as high as possible, subject to constraints and tradeoffs of other measures of performance.

Another possible measure of performance is the fraction of cases, or the fraction of radiologists' time spent reading cases, that come from a site of origin that the radiologist is associated with. Reading for a site that the radiologist is associated with is desirable, in order to help maintain the radiologist's relationship with the site. Also, physicians at a site may prefer to have their cases read by radiologists whom they know personally, and trust.

Another possible measure of performance is a measure of the quality of reading, for example based on later reviews of radiologists' reports for at least a sample of cases. The measure may be, for example, the percentage of cases for which the quality of reading was below a standard. Poor quality could be due to cases being read by radiologists who lack expertise in the appropriate area of specialization, and/or to cases being read too quickly. Determining the quality of reading of a given case, and whether it is below some standard, can be done, for example, by a peer review system or by a system which would allow referring physicians to rate reports. Optionally, a peer review module or a report rating module for this purpose is integrated with the workflow system, and/or with the PACS.

Another possible measure of performance is percentage of idle time, expressed as a weighted or unweighted average over the different radiologists, with higher idle time often being indicative of lower performance. Another possible measure of performance is global workflow, the total number of cases read, or RVUs earned, per unit time, divided by the average number of radiologists available during that time, which depends both on reading speed and on idle time.

Other possible measures of performance are measures of equitable load balancing, the equitable distribution of cases to different radiologists. Examples include a measure of the variation in number of cases read or RVUs earned per unit time by different radiologists, and/or the variation in the fraction of cases read, that match the radiologist's area of specialization among different radiologists, particularly those with the same area of specialization, or the variation in some other measure of difficulty or satisfaction among different radiologists. Ideally, these variations should be small, although some of the variation may reflect different preferences among radiologists. It should be noted that it may sometimes be possible to improve load balancing by manual assignment of some cases to radiologists, if the automatic assignment of cases by the software does not achieve as good a degree of load balancing as desired, by one of these measures of load balancing.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Exemplary Configuration of System

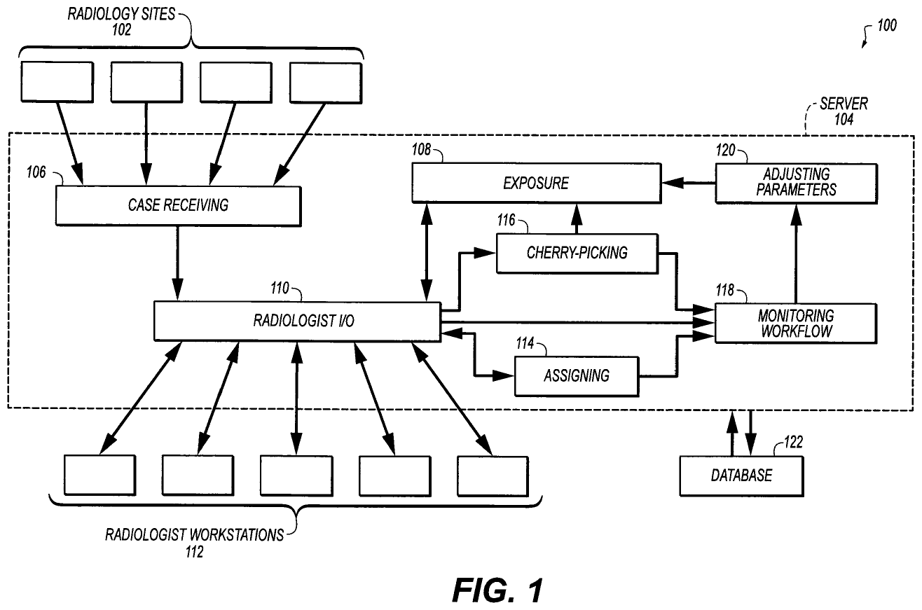

Referring now to the drawings, FIG. 1 illustrates a system 100 for assigning cases to radiologists for reading in accordance with an embodiment of the invention. Radiology cases are generated by one or more radiology sites 102, for example hospitals, clinics, and other medical facilities. The method used by system 100 may be especially useful when a plurality of different sites 102 generate cases. As used herein, a radiology site need not have a single geographic location, but “site” could refer to an organization, for example a hospital or a network of clinics, with branches at more than one geographic location. In some embodiments, each site has its own associated radiologists, who in the first instance are assigned to read the radiology cases generated by that site, but if radiologists who have a relationship with one site are overloaded, cases generated by that site can be assigned to other radiologists, as will be described below.

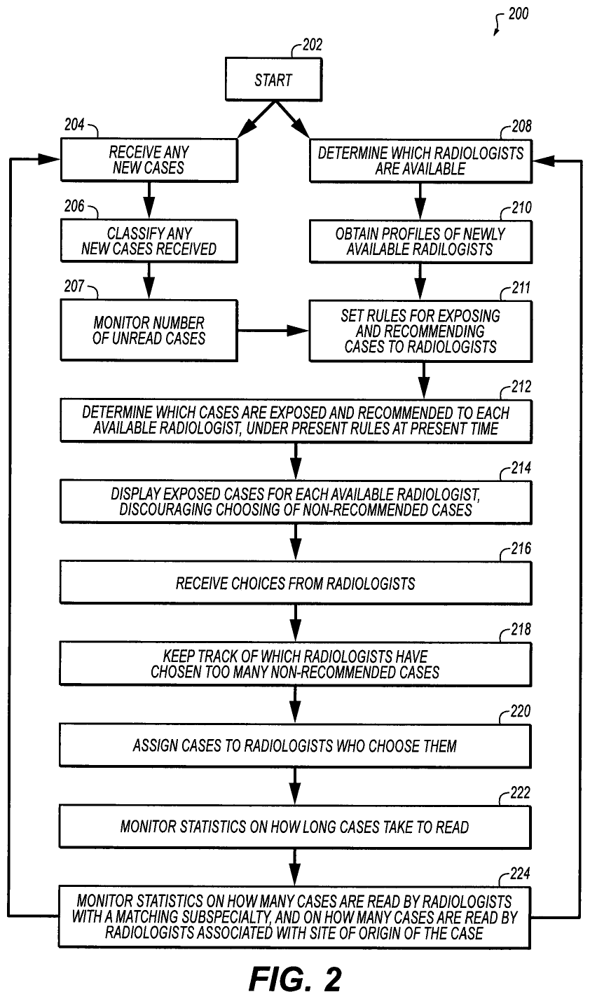

Sites 102 send their radiology cases over a communications link to a computer system 104, for example a server, running software that implements the method of assigning cases, for example as described in more detail below in the description of FIG. 2. The software is referred to as a Worklist Orchestrator program. Server 104 is shown in FIG. 1 as comprising various modules that perform different functions. The modules optionally represent different components of software, running on server 104, and/or different pieces of hardware that perform those functions, for example different computers or dedicated circuits that make up a computer system.

The radiology cases sent by sites 102 are received by a case receiving module 106 in server 104. Optionally, sites 102 send the radiology image or images for each case to receiver module 106, together with supplementary information, for example one of more of the modality, the type of case, the level of urgency, the site of origin, and special instructions such as the identity of a particular radiologist or group of radiologists that the case should be assigned to, or should preferably be assigned to. Supplementary information is commonly referred to as metadata. Optionally, the receiving module associates the supplemental information with each case in a uniform format, even if such information is sent from different sites in different formats. Associating the information with each case in a uniform format may be referred to herein as “data normalization” or simply “normalization” of the cases. The supplemental information may also include how much money will be paid to the radiologist who reads the case, expressed for example in accounting units, such as the Relative Value Units (RVUs) used by Medicare in the USA. Alternatively, the image is not sent initially to the receiver module, but only an identifier for the case, and the supplementary information, is sent to the receiver module. The image itself is then optionally communicated later directly to the radiologist who is assigned to read the case, once the case has been assigned. It should be understood that whenever an image is described herein as associated with a radiology case, there could instead be more than one image associated with the case.

Optionally, receiving module 106, another dedicated module of system 104, or software running externally that communicates with system 104, does its own preliminary analysis of the image, for example using any known method of imaging analytics, in order to flag cases that are especially urgent to read, even when this special urgency is not apparent from clinical symptoms alone. For example, in abdominal radiology cases, the imaging analytics software optionally looks for the presence of free air in the abdomen, for example by first segmenting the bowel, and then looking for voxels of low density, characteristic of air, outside the segmented bowel. In patients who have not recently had abdominal surgery, free air in the abdomen is usually an indication of a perforated bowel, which needs to be treated immediately. When the imaging analytics software detects such a feature in an image, then the case is flagged as being especially urgent. This information is optionally used by receiving module 106 in setting a deadline for reading the case, and also by exposure module 108 as will be described below, ensuring that the case will be read quickly.

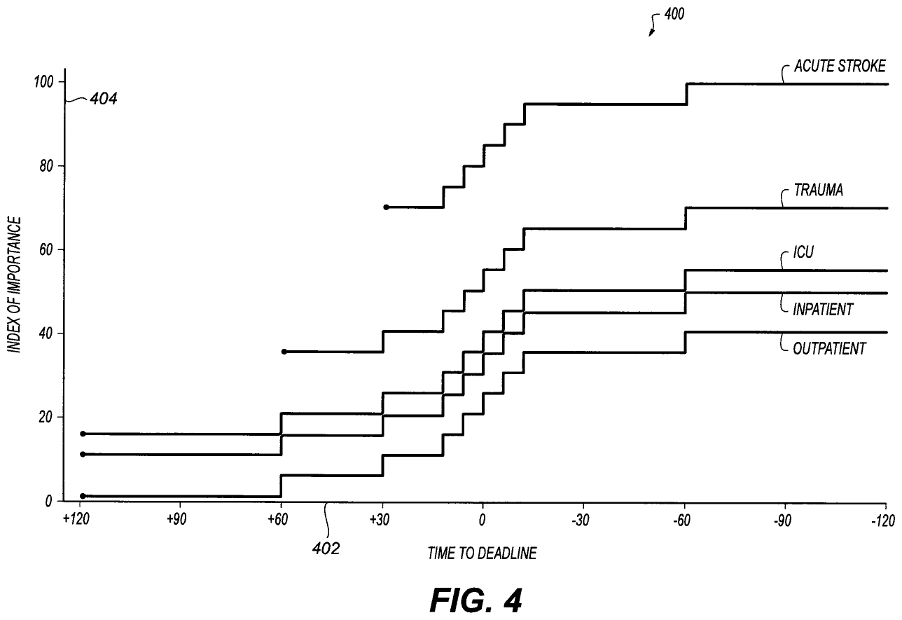

Receiving module 106, or another module of server 104, optionally associates with each received case a deadline for reading the case. Optionally, the deadline is taken directly from a deadline sent with the case from one of the sites. Alternatively, the deadline sent with the case is modified by server 104, and/or other information sent with the case, for example the level of urgency, is used to find the deadline. For example, an organization running server 104, that has contracted Service Level Agreements (SLAs) with sites 102 to read radiology cases, has a time limit for reading each case after receiving it, or after the study was done, specified in the SLA contracted with each site. The specified time limit sometimes will depend on a level of urgency assigned to each case. For example, cases coming from the most urgent cases, such as acute stroke and trauma cases, will usually have a much shorter time limit than general non-emergency cases, such as cases coming from osteoporosis patients. Receiving module 106 then uses the time limits specified in the SLA with each sites to determine the reading deadline for each case coming from that site. Even if there is no contractual SLA associated with a case, the server optionally assigns the case a deadline based on a typical SLA for that type of case. Alternatively, some or all cases are not given a well-defined reading deadline at all, but instead each of these cases is associated with an urgency for reading the case, given by a number for example, that increases with time in a certain way, and the initial urgency for reading is higher, and increases faster, for cases that require more immediate reading, such as acute stroke and trauma cases. An example of an urgency for reading a case, defined without any deadline, is the “index of importance” plotted in FIG. 4, but defined in terms of the elapsed time since the study time of the case, rather than in terms of the time to deadline.

An exposure module 108 optionally sets criteria for determining which cases are exposed to each radiologist, the exposing allowing the radiologist to choose those cases for reading. Optionally, as will be described below, these criteria need not be fixed, but in some embodiments of the invention change over time, as time goes on, and cases get closer to their deadlines, for example, without having been read. The criteria also optionally vary depending on time of day, and day of the week, since, for example, there may be fewer radiologists available to read cases during night shifts and weekend shifts, and even within a given shift, for example within the weekday daytime shift, fewer radiologists may tend to be available at certain times of day and/or certain days of the week, and radiologists may tend to work more slowly, on average, at certain times of day. At any given time, the criteria may depend on characteristics of the case, such as type of case, level of urgency, and which site the case comes from, as well as on characteristics of the radiologist, such as area of specialization, and a relationship of the radiologist with one or more of the sites.

In addition, exposure module 108 optionally sets criteria for determining which cases, exposed to a given radiologist, the radiologist will be encouraged to read, as will be described in detail below. In some embodiments of the invention, and/or in some situations, all cases are exposed to all radiologists, but different radiologists are encouraged to read different cases, for example different cases are recommended to different radiologists, or some cases are given a higher priority for reading to some radiologists, and other cases are given a higher priority for reading to other radiologists. In other embodiments of the invention, and/or in other situations, only some cases are exposed to a given radiologist, but all those cases are equally recommended for the radiologist to read.

In other embodiments and/or in other situations, only some cases are exposed to a given radiologist, and the radiologist is encouraged to read some of those cases in preference to others, for example some of those cases are recommended to the radiologist, and some are not recommended. In exemplary embodiments described below, cases are either recommended or not recommended to a given radiologist at a given time, and the radiologist is encouraged to read the recommended cases, and is not encouraged to read the cases that are not recommended. But it should be understood that in other embodiments of the invention, cases are given one of more than two levels of priority for reading by a given radiologist, and the radiologist is encouraged to preferentially choose cases with higher levels of priority over cases with lower levels of priority.

Optionally, exposure module 108 makes a list of cases that should be exposed, and cases that should be recommended, to each radiologist, and sends the list to a radiologist input/output module 110. Alternatively, exposure module 108 only sends the criteria for exposing and recommending cases to radiologist I/O module 110, and I/O module 110 receives a list of all cases and the information about them from receiving module 106, and compiles the list of cases for exposing and recommending to each radiologist, and sends the list for each radiologist to the appropriate one of radiologist workstations 112. When a radiologist logs into one of the workstations, the workstation optionally sends I/O module 110 information associating an identifier for that radiologist with an identifier for that workstation, so I/O module 110 can send the list for each radiologist to the right workstation. Alternatively, each radiologist's own workstation compiles the list of exposed and recommended cases for that radiologist, using the criteria from exposure module 108, passed through I/O module 110. Each radiologist workstation 112, when a radiologist is logged into it and is available for reading cases, displays the list of exposed cases to that radiologist, indicating which cases are recommended, at least if only some of them are recommended, and/or indicating an order of priority for the cases; Workstations 112 also optionally receive input from the radiologists on their choices of cases for reading, and optionally other input as well, for transmission to one or more modules of server 104.

### Classifying Cases

FIG. 2 shows an exemplary flowchart 200 for a method of assigning cases to radiologists that could be used by computer system 104 in FIG. 1, according to an exemplary embodiment of the invention. The method starts at 202. At 204, the receiving module optionally receives any new cases that are transferred from the radiology sites. At 206, each new case is optionally classified, optionally using information about the case that was sent by the radiology sites. Optionally, Natural Language Processing (NLP) of textual data such as a diagnosis is used to help classify cases. Classifying cases is also referred to as “data normalization” of cases, since it associates data in a consistent format with each case. In an exemplary embodiment of the invention, the following data is optionally associated with each case:
Source (Site where the study was acquired)
Patient Class (IP, OP, ED/ER/ICU)
Study Time
SLA (“Service Level Agreement”) time T_{SLA}, the time from the study time to the deadline for reading the case
Time remaining to read (Time to read=Study time+T_{SLA}−present time)
Acute stroke (Yes, No)
Trauma (Yes, No)
Flagged as urgent by imaging analytics (Yes (and reason), No)
Priority (STAT, Urgent)
Subspeciality, also referred to herein as “specialty” or “service area.” A case may be associated with more than one subspeciality, for example a head scan of a child would be associated with both “pediatric radiology” and “neuroradiology.”
Ordering Provider (Referring Physician)
Modality
RVU (“Relative Value Unit”)
Procedure Code\Description, for example the CPT code for the procedure
Patient Location within the hospital (department the patient is hospitalized in)

Each case is assigned to a level of urgency that depends on its associated data.

There are one or more levels or urgency, each with one or more types of cases assigned to it. In an exemplary embodiment of the invention, the levels of urgency, and the categories of cases assigned to each level, are as follows. These levels are sometimes referred to as “buckets.”

Level 1: Acute stroke and trauma. Alternatively, only acute stroke cases are included in Level 1, and trauma cases are included in Level 2, or trauma cases are included in their own level, below acute stroke cases but above the cases listed below as Level 2, which would then be called “Level 3.”

Level 2: Emergency Department (ED)/Emergency Room (ER), Intensive Care Unit (ICU), Urgent (STAT)

Level 3: In-patient (IP)

Level 4: Out-patient (OP)

Level 5: General. This is a catch-all category, for example for cases for which there is inadequate information to classify it, and generally will have few or no cases in it.

Sometimes, a case may fall into more than one of these categories. In that situation, the case is optionally assigned to the highest level of urgency that corresponds to any of its categories. However, its other categories may also be used, for example for purposes of assigning it to an appropriate specialist if possible.

Optionally, if a radiologist sees a case that appears to be incorrectly classified, and in particular if the case appears to be designated with too low a level of urgency, then the radiologist's workstation has the ability to reclassify the case to its correct category and level of urgency.

Imaging modality includes, for example, CT, CR (conventional x-ray image), MRI, and ultrasound. Procedure description includes the presence or absence of contrast agents, the type of contrast agent if any, and the part of the body that is imaged, for example head, chest, abdomen, or limbs. These classifications are potentially important because different radiologists may specialize in reading different types of images, and it may be more efficient to have a case read by a radiologist who specializes in reading that type of image. Source is a piece of data that is optionally used in assigning cases, since sites may prefer to have their cases read by radiologists who have a relationship with them, either physically located at that facility, or working for them remotely. The SLA time interval T_{SLA}, within which the case is to be read, typically depends on the level of urgency of the case, and may also depend on the site that the case comes from, for example if the organization that is assigning cases to radiologists for reading has different Service Level Agreements with the different sites, specifying the time intervals within which cases are to be read. It should be noted that, in some embodiments of the invention being described, cases are given a well-defined deadline for reading, and this is the usual practice in hospitals and clinics. However, other ways of assuring timely reading of the case are possible, for example by associating the case with a numerical urgency for reading that increases with time, giving the case an increasingly high priority for reading, even without a well-defined deadline.

Other data that may be associated with each case, even if it is not used in assigning cases, includes a unique patient ID number, and patient name, age, and gender. Cases may also be designated, by the site that sends them, for assignment preferably to a particular radiologist or group of radiologists, for example a radiologist or a group that works for that site, on location or remotely, and this information is optionally associated with the case when it is received.

Optionally, if a case is flagged by imaging analytics as having a medical condition or feature that makes it especially urgent to read, then it is assigned to level 1, even if it was assigned to a lower level based on information from the site of origin. Alternatively, it is assigned to a level that depends on which feature was found by imaging analytics, whether or not that level is higher than its original level, but optionally it is assigned to a level no lower than its original level.

At 207, a module 118 for monitoring workflow, shown in FIG. 1, optionally monitors the number of unread cases, optionally sorted by category of level of urgency, by subspecialty, by site of origin, and/or by other factors that may affect which radiologists are best suited to read a case. In FIG. 1, monitoring module 118 is shown receiving this information from radiologist I/O module 110, which would have information about the number and classification of unread cases, but alternatively the information comes directly from case receiving module 106 and/or from exposure module 108. This information, and other information that monitoring module 118 collects about the performance of the system, is optionally used as input for making adjustments to rules used for determining which cases will be exposed and/or recommended to which radiologists, as will be described below.

### Radiologist Profiles

At 208, in parallel with or before or after receiving and classifying new cases, radiologist I/O module 110 determines which radiologists are currently logged in and available for reading cases. At 210, profiles for any newly available radiologists are optionally obtained. These profiles, which may be stored in a database that the radiologist I/O module or the exposure module has access to, may include any of the following information, which sometimes affect which cases a radiologist is exposed to:

1) One or more sites that the radiologist has a relationship with, for example working at the physical location of the site, or working for the site remotely (referred to as “virtual location”) as an employee or as a contractor. Also, optionally, particular attending physicians that the radiologist has a relationship with, who may prefer that the radiologist reads their cases. Some radiologists may be authorized to do work for any site, and do not have a preferred site.

2) One or more groups, if any, that the radiologist belongs to within the site, for example “conventional x-ray readers.”

3) One or more areas of specialization of the radiologist, for example regions or functions of the body such as neurology, gastroenterology, musculoskeletal, or cardiology; medical conditions; characteristics of the procedure such as contrast agent use; and imaging modalities, which the radiologist is particularly trained to read.

4) The usual shift that the radiologist works.

5) Reading credentials of the radiologist, for example the radiologist may only be legally qualified to read certain types of cases, or to read cases from certain sites or for certain groups within a site.

6) Role of the radiologist, for example resident, fellow, or attending physician, which may make certain cases more appropriate for the radiologist to read.

Some of the above information may not be applicable to some radiologists. Other potentially relevant information about the radiologist may be included in the profile as well.

### Exemplary Criteria for Exposing Cases Over Time

At 211, rules, also referred to as criteria, are optionally set for determining which cases will be exposed and which cases will be recommended to which radiologists. Optionally, the rules also determine a relative order of priority of different cases that are exposed to the same radiologist. Optionally, these rules are not fixed, but, for example, have one or more parameters that can be adjusted, for example automatically by the system and/or manually by an administrator. The rules may be useful to improve performance of the system, for example in response to information about the performance of the system that is monitored by monitoring module 118. The one or more adjustable parameters include, for example, parameters that determine when cases in different categories are escalated, such as the parameters P1 and P2 described below, and/or parameters that determine how many cases in different categories will be recommended to each radiologist, and/or how many cases in different categories each radiologist will be allowed to choose.

FIG. 1 shows an adjusting module 120 that adjusts these parameters automatically, in response to the monitored performance information. For example, if there is a larger than usual number of unread cases in a given category or a given subspecialty, or if the number of such unread cases is increasing in time, then optionally the escalation parameters for cases in that category or subspecialty are adjusted so that those cases are escalated earlier, exposing them to more radiologists and making it likely that they will be read sooner. Adjusting module 120 communicates the adjusted parameters to exposure module 108. Optionally, adjusting module 120 uses a feedback mechanism, for example a feedback algorithm known from control theory, in order to adjust the parameters in response to specific measures of performance obtained from monitoring module 118, such as the number of unread cases in different categories and different subspecialties and from different sites, and any of the measures of performance described below in the section headed “Performance monitoring.” The feedback algorithm optionally finds new values of the parameters that it predicts, based for example on past experience, will result in improved values of the measures of performance. Optionally, adjusting module 120 attempts to maximize a measure of overall performance, for example a measure representing a desired tradeoff between how quickly cases are read, and how many cases are read by matching subspecialists and radiologists associated with the site of origin. Alternatively, adjusting module 120 attempts to set a plurality of different measures of performance as close as possible to values that are considered to be optimal. Optionally, system administrators can adjust how the measure of overall performance is defined, and/or the optimal values of the different measures of performance, depending on local conditions and the needs of the users of the system.

At 212, for each available radiologist, exposure module 108 optionally applies the rules about exposing and recommending cases, determining which cases will be exposed and which cases will be recommended to that radiologist at that time. In an exemplary embodiment of the invention, the following three rules govern the exposure of cases: 1) Level 1 cases are to be exposed to all available radiologists. 2) During a weekday daytime shift, cases from Levels 2-5 initially are to be exposed only to available radiologists whose area of specialization matches the type of case, and who are associated with the site that the case comes from. 3) During a weekend or holiday shift, or during a weekday night shift, cases from Levels 1-3 are to be exposed to all available radiologists, and cases from Levels 4 and 5 initially are to be exposed only to available radiologists whose area of specialization matches the case, and who have a relationship with the site that the case came from.

When applying these rules at 212 to determine which cases to expose to each radiologist, the following exception to the rules optionally occurs for cases that are designated for assigning only to a particular radiologist or group, in some embodiments of the invention. Optionally, those cases are initially exposed to that radiologist or group, and only to that radiologist or group. Optionally, a case may be designated for assigning only to a particular radiologist or group by its originating site, in which case that information is associated with the case when it is received and classified. Optionally, a case may be manually designated for assignment to a particular radiologist by an administrator or technician, for example in order to achieve better load balancing, if the administrator feels that the software is not achieving satisfactory load balancing in its automatic assignment of cases to radiologists. Optionally, a case may be manually designated for assignment to a radiologist by that radiologist, by exercising an “assign to me” option, who may believe that he or she is the radiologist best able to read the case, perhaps because the radiologist has recently read similar cases, perhaps from the same patient. Optionally, a radiologist may manually designate a case for assignment to another radiologist or group, who the designating radiologist thinks is the most appropriate radiologist or group for reading that case. Optionally, if a radiologist manually designates a case for assignment to someone else, then cherry-picking module 116 makes a determination of whether the radiologist did this for selfish reasons, to avoid having to read an undesirable case, for example by evaluating how undesirable the case is, and whether it is plausible that the designated radiologist is particularly qualified to read it, and cherry-picking module 116 takes such selfish behavior into account in deciding whether to consider the radiologist a cherry-picker.

As a case gets older without being read, the rules about exposing and recommending cases, the rules established at 211 may specify that the case is exposed to more radiologists. In addition, in some circumstances other less urgent cases may be exposed to fewer radiologists, to make it more likely that the more urgent cases will be read by the deadline. Specifically, in some embodiments of the invention, the exposure criteria change for cases that have reached an escalation time. In general, there may be more than one escalation time associated with a case, for example there may be a first escalation time, after which the case is exposed to more radiologists, and a second escalation time, after which the case is further escalated, exposing it to still more radiologists.

In an exemplary embodiment of the invention, a case has up to two escalation times. The first escalation time occurs at a time interval T1 after the case is received. The second escalation time occurs at a time interval T2 after the first escalation time. The time interval from the second escalation time to the deadline for reading the case is designated as T3, so the total time interval for reading the case, specified for example in the SLA, is T_{SLA}=T1+T2+T3. In general, T1, T2 and T3 are each a certain fraction of the total time interval T_{SLA}, T1=P1*T_{SLA}, T2=P2*T_{SLA}, and T3=P3*T_{SLA}, with P1+P2+P3=1, and with P1, P2, and P3 depending in general on the level of urgency of the case, and on the shift. During time interval T1 after the case is received, up to the first escalation time, a case is exposed only to available radiologists whose area of specialization matches the case, and who have a relationship with the site that sent the case. During the time interval T2, between the first and second escalation time, a case is exposed to all radiologists whose area of specialization matches the case, even if they do not have a relationship with the site that sent the case. During the time interval T3 between the second escalation time and the deadline, and after the deadline if the case has still not been assigned for reading, the case is exposed to all available radiologists, regardless of their area of specialization and regardless of whether they have a relationship with the site that sent the case. However, a case that is designated for assigning only to a particular radiologist or group of radiologists is exposed to that radiologist or group, and only to that radiologist or group, at least up to the first escalation time, and optionally also up to the second escalation time, and optionally also after the second escalation time. In an exemplary embodiment of the invention, such cases are exposed to all available radiologists after the first escalation time, for Level 2 cases, and they are exposed to all available radiologists after the second escalation time, for Level 3, 4, and 5 cases, while Level 1 cases are always exposed to all available radiologists. It should be noted that for those levels of urgency and shifts for which cases are initially exposed to all available radiologists, as described above, in effect P1=P2=0, so the second escalation time is the time that the case is received, and the case is not escalated after that.

In an exemplary embodiment of the invention, the values of P1, P2, and P3 shown in Table 1 are used, as a function of level of urgency, for the day shift.

TABLE 1
Values of P1, P2, and P3 for day shift

| | Level 1 | Level 2 | Level 3 | Level 4 | Level 5 |
|---|---|---|---|---|---|
| P1 | 0 | 0.3 | 0.4 | 0.4 | 0.4 |
| P2 | 0 | 0.3 | 0.35 | 0.35 | 0.35 |
| P3 | 1 | 0.4 | 0.25 | 0.25 | 0.25 |

In another embodiment of the invention, P1=0.75 and P2=0 for Levels 3, 4, and 5, so in effect there is only a single escalation time for Levels 3, 4, and 5. For weekend, holiday, and night shifts, the values are the same as in the day shift, for Levels 1, 4 and 5, but P1=P2=0 and P3=1 for Levels 2 and 3, reflecting the fact that fewer radiologists are working during the weekend, holiday and night shifts, so it may be better to expose even Level 2 and 3 cases to all available radiologists from the beginning, with the goal that they will be read sooner, and to try to ensure that they are read by the deadline. In some embodiments of the invention, the P1, P2, and P3 parameters are configurable, and can either be set to the typical default values mentioned above, or to different values, at the time of system installation, or later, as the system is tuned in the field to optimize performance. In other embodiments, the parameters are adjusted dynamically by the system as time passes, based on real time monitoring of system performance, historical data acquired from monitoring workflow and system performance, and/or a feedback mechanism.

In addition to exposing escalated cases to more radiologists, cases that have not been escalated, or are less escalated, are optionally hidden from exposure to radiologists who are exposed to too many escalated cases. For example, in an exemplary embodiment of the invention, there is a maximum number of cases that a radiologist is exposed to at any given time, for example 12 cases, and only the 12 highest priority cases are exposed to that radiologist, if there would otherwise be more than 12 cases that the radiologist is exposed to. Such a rule can be used whether cases in different categories are displayed in separate sub-worklists or are displayed in a single merged worklist, but either way the existence of such a rule presumes that there is a definition of relative order of priority for cases in different categories, for example as described above in the section headed “Setting an overall order of priority for cases from different categories”. Alternatively, there is a maximum number of cases that a radiologist is exposed to within a category of cases, for example there is a maximum number of acute stroke cases, and/or a maximum number of trauma cases, that a radiologist is exposed to. In some embodiments of the invention, the maximum number of cases, and/or the maximum number within a category, is changed dynamically over time, by an administrator or automatically by the system, in response to measures of workflow efficiency, in order to improve the workflow efficiency.

Optionally, the highest priority cases, at least within a given category, for a workflow system where there are two escalation times, are the ones that are past the second escalation time, followed by those that are between the first and second escalation time, followed by those that are before the first escalation time. Among cases that have reached a given escalation time, higher priority is optionally given to those cases with less time remaining before the deadline for reading. Cases that are past their deadline, and still haven't been read, are considered to have a negative value of time remaining, and have a higher priority. For cases that have the same time remaining, higher priority is optionally given to cases that match the area of specialization of the radiologist. For cases that have the same time remaining, and that all match or all do not match the area of specialization of the radiologist, higher priority is optionally given to the cases that come from a site that the radiologist has a relationship with. In practice, since two cases are not likely to have exactly the same time remaining, the order of priority of the cases listed for a given radiologist is often based only on the time remaining.

The values of P1, P2, and P3, for different levels of urgency and for different shifts, may be adjusted from time to time by system administrators, in order to improve the efficiency of the method. For example, the values maybe be changed on a trial basis for a while, and if this results in a better outcome, measured for example by fewer cases failing to be read by their deadline, by fewer cases being read by non-specialists, and/or by greater overall workflow per radiologist, then the new values are kept, while if the change results in a worse outcome, then the new values are rejected in favor of the old values.

### Exemplary Criteria for Recommending Cases

An exemplary procedure for the workflow system to determine which of the exposed cases are to be recommended to a radiologist, at 212 in FIG. 2, is to determine an order of priority for the exposed cases, in each category of level of urgency, and to specify that only a fixed number N of the highest priority cases in each category are recommended to the radiologist, for example, only the first 2 cases, or the first 3 cases, or the first 4 cases. If there are fewer than N exposed cases in a given level, then the workflow system recommends all of those cases to the radiologist. The priority is optionally higher for cases that have less time remaining before the deadline, and typically, escalated cases have less time remaining than cases that haven't been escalated yet, so the escalated cases are often the ones with the highest priority. For cases that have the same time remaining, higher priority is optionally given by the workflow system to cases that match the area of specialization (also called subspecialty) of the radiologist. For cases that have the same time remaining, and that all match, or all do not match, the area of specialization of the radiologist, higher priority is optionally given to the cases that come from a site that the radiologist has a relationship with. In practice, since two cases are not likely to have exactly the same time remaining, the order of priority is often based only on the time remaining. Alternatively, the workflow system gives all escalated cases higher priority than it gives any non-escalated cases, even if the escalated cases do not have less time remaining.

Optionally, the numbers of recommended cases N, for each category, are among the parameters adjusted by adjusting module 120, in response to measures of performance monitored by monitoring module 118, at 211. In some embodiments of the invention, there is a fixed number of cases exposed to each radiologist, for each category, and these numbers are adjusted by adjusting module 120. An example of circumstances in which these numbers are adjusted is given below, at the end of the section labeled “Cherry-picking.” Optionally, if the number of recommended cases, or the number of exposed cases, is limited in one or more categories, because there are other categories where cases are not being read soon enough, then measures of performance for the different categories, for example the percentage of cases read by their deadline, or read by 80% of the SLA time interval, are displayed to the radiologists. By seeing these measures of performance, the radiologists may better understand why they are being limited in the number of cases exposed or recommended to them, and they may feel less resentful and more willing to do their share to solve the problem.

It should be understood that the rules for exposing cases to radiologists, the rules for the order of priority given to cases, the rules for when cases are escalated, and other features of the workflow system described above, are only exemplary and only refer to some embodiments of the invention. Other rules and other features are optionally used in different configurations of the system, and in different embodiments of the invention.

### Display of Cases

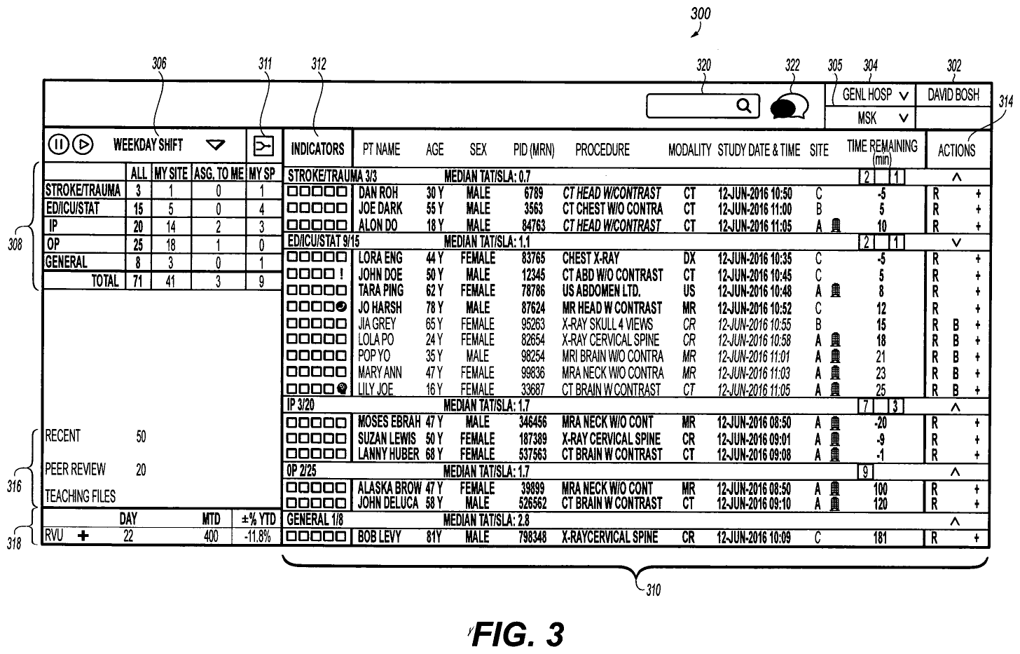

At 214, information about the cases currently exposed to each available radiologist is displayed in a worklist on the workstation that that radiologist is using, optionally with an indication of which displayed cases are recommended and which are not, and/or with an indication of an order of priority for reading the cases. Optionally, the cases are grouped together by category of level of urgency, and within each category, the cases are listed from top to bottom in order of priority, with the recommended cases listed higher up. The cases grouped together in each category are referred to as a “sub-worklist.” FIG. 3, to be described below, shows an example of such a display, in accordance with an exemplary embodiment of the invention. The display includes user interface elements, such as buttons for clicking on or touching, that allow a radiologist to choose one of the exposed cases for reading. The display optionally also includes user interface elements, such as a drop-down menu, for specifying a reason if a non-recommended case is chosen.

Optionally, if the cases are grouped into sub-worklists, the order of priority of the cases within each sub-worklist is set by the workflow system based on the time remaining until the deadline for reading the case, with cases that are closer to their deadline having higher priority. Additionally or alternatively, other factors or combinations of factors are used by the workflow system in determining the order of priority of the cases. For example, imaging analytics is optionally is used by the workflow system, after cases are received at 204, to determine whether an image from the case exhibits one or more features that indicate that the case is especially urgent for reading, even if this urgency is not apparent from clinical symptoms, and from information associated with the case when it was sent by the site of origin. An example of such a feature would be the presence of free air in the abdomen, which may be easy to detect by image processing, and which may indicate a perforated bowel, which requires immediate treatment. If imaging analytics is used, or if a preliminary reading of the case is made by a person then optionally, any case that is found to exhibit such a feature is moved to a higher order of priority than any cases that do not exhibit such a feature, or at least to a higher order of priority than cases that do not exhibit the feature and have the same or almost the same deadline.

Optionally, the cases are not divided into sub-worklists, but information about the cases in different categories is displayed in a single merged worklist. In this situation, optionally the order of priority for the merged worklist is not based simply on the time remaining to deadline, but on an index of importance that depends on the time remaining to deadline (including negative time to deadline when it is past the deadline), as well as on the category of the case, and optionally on other characteristics of the case, to take into account that for two cases in different categories with the same time to deadline, it is generally more important to read the one in the category with a higher level of urgency. Exemplary algorithms for calculating the index of importance are described below in the section headed “Exemplary algorithms for index of importance.”

Within a given sub-worklist, cases that have been escalated generally have less time to deadline than cases that have not been escalated, and will be listed at the top of the sub-worklist. As noted for example with reference to FIG. 3, information displayed about cases optionally includes the subspecialty and site of origin. If so, then by looking at which cases are listed near the top of a sub-worklist, a radiologist can see at a glance whether one site, and/or one subspecialty, is falling behind in reading their cases, because that site and/or subspecialty will have a greater than normal number of escalated cases.

### Cherry-Picking

At 216, radiologist I/O module 110 receives information about which cases the radiologists chose for reading, and, in some embodiments of the invention, receives any reasons that the radiologist submits for choosing a case that is not recommended, or that is lower priority than other cases that could have been chosen first. Choosing a non-recommended case is referred to as “breaking glass,” an expression which indicates that this action is logged by the system. Such choosing of lower priority cases first, with only selfish motivations, and without an approved reason, is referred to as cherry-picking. Radiologist I/O module 110 optionally passes these reasons on to cherry-picking module 116. Optionally, the workflow system presents the radiologist with a menu listing possible reasons, and the radiologist selects a reason from the menu, or enters a different reason manually. Reasons that are optionally listed on the menu, and that would be considered approved reasons for choosing a non-recommended or lower priority case, include 1) the radiologist does not feel qualified to read any of the recommended cases that she or he was exposed to, and 2) the radiologist believes, for clinical reasons, that the chosen case should have higher priority than any of the recommended cases. Optionally, the specified reasons are saved by cherry-picking module 116 and later made available for viewing, for example by the radiologist's supervisor, to determine whether the specified reason was valid or not.

At 218, cherry-picking module 116 keeps track of the occasions on which each radiologist chooses a non-recommended case, or a too low priority case, and logs any reason provided by the radiologist. Choosing a non-recommended case without an approved reason is defined herein as an instance of cherry-picking.

Optionally, if a radiologist engages in more than a threshold number of instances of cherry-picking, within a specified time interval or out of a specified total number of cases read, as recorded by cherry-picking module 116, then module 116 informs exposure module 108 of the identity of the radiologist, and exposure module 108 changes the rules for which cases that radiologist is exposed to, exposing the radiologist to less desirable cases than radiologists who have not done as much cherry-picking. This change in the rules for exposing cases is performed at 211, as described above. Additionally or alternatively, cherry-picking module 116 makes the extent of the cherry-picking of that radiologist, or of all the radiologists, known to the other radiologists, which may motivate radiologists not to engage in cherry-picking because of peer pressure.

Cherry-picking module 116 optionally passes information about cherry-picking on to monitoring module 118, which uses the information to compile statistical measures of how much cherry-picking is going on among the radiologists in general, or among different groups of radiologists, for example radiologists in different subspecialties. Alternatively, monitoring module 118 obtains this information directly from radiologist I/O module 110. Alternatively or additionally, monitoring module 118 obtains information at 218, for example from cherry-picking module 116 or directly from radiologist I/O module 110, about radiologists who are choosing lower priority cases before higher priority cases, even if the lower priority cases are also recommended, and compiles this information into statistical measures about how many radiologists are doing this. These measures of performance are optionally used by adjusting module 120, to adjust parameters in the rules for exposing and recommending cases, in order to improve performance of the system. For example, if one of these measures of performance shows that too many radiologists are choosing lower priority but still recommended cases before higher priority cases, in one or more categories, then optionally the number of recommended cases N, in those categories, is reduced. Assuming radiologists are inhibited from choosing non-recommended cases, this change in the rules is likely to reduce the number of radiologists choosing lower priority cases before higher priority cases. Furthermore, the number of exposed cases in those categories is optionally reduced, in response to radiologists choosing too many lower priority cases, including non-recommended cases.

### Assignment of Cases

At 220, assigning module 114 records that cases have been assigned for reading to radiologists who chose them, and radiologist I/O module 110 informs the radiologists of the cases that were assigned to them. Assigning module 114 may use various rules to decide which radiologist to assign a case to, if it is chosen by two or more radiologists at the same time, or within a short time interval of each other. For example, the case may preferably be assigned, in that circumstance, to a radiologist whose area of specialization matches the type of case, and/or to a radiologist who has a relationship with the site that the case came from, and/or to a radiologist who is behind in an expected quota of cases to be read that month, or who is behind in an expected quota of “Relative Value Units,” or a similar accounting unit used for paying radiologists, for the cases to be read that month. Assigning module 114 optionally informs exposure module 108 when cases have been assigned, and cases that have already been assigned are optionally not included among the cases exposed to other radiologists, or the radiologists whom they were assigned to, by exposure module 108, at 212.

The radiologist who was assigned the case can now access the one or more images of the case, through radiologist I/O module 110, or directly from the site that sent the case, for example by referring to an identifier for that case. After reading the case, the radiologist can send a report back to that site, directly or through radiologist I/O module 110. Once a case has been assigned at 220, the functions of displaying the image to the radiologist, and receiving the radiologist's report, need not be part of the workflow software defined by flowchart 200 at all, but a window is optionally opened on the radiologist's work station controlled by separate PACS software, which handles the functions of displaying the image and optionally the referring physician's diagnosis to the radiologist, providing a user interface for the radiologist to write the report, and receiving the radiologist's report and storing it in the PACS. Optionally, the PACS software is conventional software that came with the PACS system, not designed specifically to run with the workflow software of flowchart 200, but the workflow software is designed to be integrated with the software of the PACS system.

In some embodiments of the invention, there is a provision in the workflow system for forcibly assigning some cases to at least some radiologists, at 220, even if they haven't chosen the cases for reading. This is done, for example, with cases having characteristics that tend are known to be considered especially undesirable, and that radiologists tend not to choose. This may be true, for example, of some routine conventional x-ray cases, such as cases involving broken bones, that are easy to read but that pay very little money, and that are of a low enough level of urgency that there are almost always higher priority cases ahead of them in a radiologist's worklist. Optionally some cases are designated in advance for such forced assignment, based on their characteristics, and possibly on the number and characteristics of currently logged in radiologists, which make it likely that these cases will not be voluntarily chosen for reading. Additionally or alternatively, cases are designated for forced assignment because they have gone unread for a specified time interval that may depend on characteristics of the case, and possibly also on the number and characteristics of currently logged in radiologists. Once a case has been designated for forced assignment, it is assigned to one of a group of radiologists who have been designated to read such cases. This group may be defined, for example, as radiologists who have a subspecialty of “general,” and/or radiologists who have worked fewer hours than expected that month, and/or radiologists who have read less than a fair share of undesirable cases, defined in some way based on characteristics of the cases. These cases are assigned to radiologists in the group in a fixed order, or randomly, or giving preference to radiologists who have worked fewer hours or who have read fewer undesirable cases, or according to some other formula. Optionally, even after a case has been forcibly assigned to one radiologist, it is still listed on the worklists of other radiologists until the assigned radiologist actually reads it, and if another radiologist chooses it for reading first, then the assigned radiologist does not have to read it. That may be useful particularly for cases that are close to their deadlines, in order to increase the likelihood that the case will be read before its deadline. But if no other radiologist chooses it for reading, then the radiologist that it has been forcibly assigned to is expected to read it, for example before reading any other cases, or before logging off the system.

### Performance Monitoring

At 222, after the case has been assigned, monitoring module 118 optionally receives information, for example from assignment module 114, about how long it took the case to be chosen and assigned after it was received or after the time of the study, and uses that information to compile a statistical distribution, expressed for example as a histogram, of how long it takes for cases to be assigned, optionally sorted by category, by subspecialty, by site of origin, and/or by other classification data. This statistical distribution, and some of the other statistical distributions described herein as measures of performance, are optionally calculated over a time window that changes every time they are calculated. They are based, for example, on all cases that were assigned during a specified preceding time period. Optionally, the time period is long enough compared to the time between successive calculations so that the time windows from consecutive calculations have substantial overlap, for example more than 50%. Additionally or alternatively, monitoring module 118 compiles a statistical distribution of how long it takes cases to be read and the radiologist's report submitted, after they have been received or after the time of the study. Additionally or alternatively, monitoring module 118 compiles a statistical distribution of how long currently unread cases have been waiting to be read. Optionally, monitoring module 118 uses the statistical distribution to derive a single number, or a few numbers, representing one or more measures of performance of the system, for example a mean or median time required for cases to be chosen and assigned from when they were received or from when the study was done, a mean or median age of still unread cases, and/or the fraction of cases, for example cases whose deadline fell within a specified preceding time interval, that were not assigned by their deadline. The statistical distribution and/or the measures of performance are optionally used, together with any other measures of performance compiled by monitoring module 118, by adjusting module 120, to adjust parameters of the rules for exposure and recommendation of cases, at 211. For example, if cases in one or more categories or subspecialties are taking too long to read on average, or if too many of them are missing their deadline or almost missing their deadline for reading, then optionally the parameter P1, or P1+P2, is made smaller, for those categories or subspecialties, so that those cases are escalated earlier, and are likely to be read sooner.

At 224, monitoring module 118 optionally receives information, for example from assignment module 114, about whether the case was assigned to a radiologist with a subspecialty that matches the case, and/or information about whether the case was assigned to a radiologist associated with the site of origin of the case. Monitoring module uses this information to compile a statistical measure of what fraction of cases are being assigned to radiologists with subspecialties that match the case, and/or a statistical measure of what fraction of cases are being assigned to radiologists associated with the site of origin of the case, optionally sorted by category, by subspecialty, by site of origin, and/or by other classification data. These statistical measures, which can serve as measures of performance of the system, are optionally used by adjusting module 120, to adjust parameters of the rules for exposing and recommending cases to radiologists. For example if too large a fraction of cases, in one or more categories or subspecialties, is being assigned to radiologists who do not have a matching subspecialty, and if on average those cases are being read well before their deadline, and few or none of them are missing their deadline, and the number of unread cases is not too large and not increasing, then optionally the parameter P1, or P1+P2, is increased, for those categories of subspecialties, so those cases are escalated later, and they are more likely to be read by radiologists who have a matching subspecialty.

The measures of performance found by monitoring module 118 are also optionally available to system administrators, who can use the information to manually adjust rules for exposing and recommending cases to radiologists, or to adjust other system parameters, with the goal of improving system performance according to some definition.

The method then returns to 204, receiving new cases, and 208, determining which radiologists are available, followed by 210, which obtains the profiles of the newly available radiologists from the profiles stored in the system database and any selections they may make at login, and the loop may continue indefinitely. It should be understood that in flowchart 200, paths shown in parallel can be done in parallel, or serially in any order, and some actions in the overall loop could be done in a different order. For example, the monitoring actions at 207, 222 and 224, which accumulate information about many cases, can be done at any place in the loop.

### Removal of Unread Cases

In some embodiments of the invention, there is no expectation that all cases will eventually be read, and the loop in flowchart 200 optionally includes a provision for removing cases that have not been read for a relatively long time, for example at a certain time interval past their deadlines, and are in a category of a relatively low level of urgency. This is done, for example, after 224, before returning to 204 and 208, or at any other point in the loop. Such a provision may be used, for example, if it is known that on average there are not enough radiologists in the system to read all radiology cases received, in the long run, and it is desired to remove less urgent cases so that the number of cases will not increase indefinitely. Optionally, cases that appear likely never to be read have their order of priority gradually decreased relative to other cases, until they are finally removed completely, as described above near the end of the section headed, “Setting an overall order of priority for cases from different categories.” Alternatively, these cases have their order of priority increase, at least relative to other cases in the same category, as they go further past their deadline, until they are removed. It should be noted that even if some cases are never read as part of the workflow system, their image files are still optionally available for viewing by medical personnel who are providing care for the patients concerned.

### External Database

Optionally, all data received by or generated by any of the modules in server 104, for example in performing any of the procedures shown in flowchart 200, is stored internally in a memory of the server. Alternatively, an external database 122, with a communications link to server 104 and its modules, stores some or all of this data, and optionally some or all of the data is stored both in the memory of the server and in the database. The data includes, for example, one or more of the following:

1) image files for the different cases, if the image files are received by the server;

2) data for classifying cases, generated by data normalization;

3) criteria for exposing and/or recommending cases to different radiologists;

4) the worklists of the radiologists, optionally including an order of priority of the cases listed, and/or which cases are recommended;

5) the identities of radiologists who have been assigned to read the cases;

6) data on cherry-picking by different radiologists;

7) reports by the radiologists who have read the cases;

8) other data associated with cases, including preliminary reports and notes;

9) performance monitoring data generated by the system.

Storing this data both in the memory of the server and in an external database has the potential advantage that the data will be backed up, and the system can quickly be restored if the server crashes, preventing long delays in reading cases which could be life threatening.

If the image files are received and saved by the server, either in its own memory or in database 122, then optionally the images are analyzed later, for example for administrative purposes, even if imaging analytics is not used in real time to flag cases that are more urgent than expected. Even if imaging analytics is used initially in real time, later analysis of the images, possibly only a portion of the images selected on the basis of the initial imaging analytics, can be used for example for more computationally intensive imaging analytics that would take too long to do in real time initially. This is done, for example, for ongoing checking of the quality of the initial imaging analytics. Using an external database to store the image files for this and/or other purposes has the potential advantage that an external database is likely to have more storage capacity than the memory of the server, which may be needed for storing a large number of image files.

### Exemplary Display to a Radiologist

FIG. 3 shows an example of a display 300 that might be seen by a radiologist user, who has logged into a workstation, using the method of FIG. 2 and the system of FIG. 1, according to an exemplary embodiment of the invention. FIG. 3 also illustrates a few optional features of the workflow system not described elsewhere herein, for example the “Hotline” feature described below. It should be understood that, in the description of FIG. 3, statements about the format of the display are all optional, and are only meant to describe the choices made in one exemplary embodiment of the display. In addition, the particular information shown in the display in FIG. 3, such as the name of the radiologist, the names of patients, the descriptions of procedures, and the dates and times, are all fictional, and are only intended to illustrate how the display might appear at a given time in a given situation. The name 302 of the user, the radiologist who is logged in, is shown in the upper right corner of the display for example. An identifier 304 of the site that the user is working for, is shown to the left of name 302. A user may have a relationship with more than one site, and if so, a drop down menu attached to identifier 304 optionally allows the user to select a different site or more than one site. Optionally, for those cases that are exposed only to radiologists associated with the case's site of origin, a case will be exposed to a user only if it originates from the site that the user is logged in under, and not if it originates from other sites that the user is associated with.

A subspecialty identifier 305, for example just below site identifier 304, shows the subspecialty that the user has expertise in, initially the user's default subspecialty, for example “MSK” for “musculoskeletal.” Optionally, the user can use a dropdown menu with indicator 305 to select a different subspecialty. For example, if a user who has some expertise in a different subspecialty might wish to see unescalated cases matching that subspecialty if there is a shortage of radiologists logged in with expertise in that subspecialty. Optionally, if the user has expertise in more than one subspecialty, the menu allows the user to select more than one subspecialty at a time.

A menu 306 at the upper left of display 300 shows the default shift of the user, for example “Weekday Shift,” “Night Shift” or “Weekend and Holiday Shift.” If the user is logging in to work a different shift, then the drop down menu can be used to change to that shift. Optionally, if the rules for exposing and recommending cases are different for different shifts, then the rules are applied based on what shift the user has logged in under, even if it is outside the normal hours for that shift. Alternatively, the rules are applied based on what shift is nominally on duty at that time, even if the user logged in under a different shift. Clicking on the “Play” button on menu 306 indicates that the user is available for reading cases. Clicking on the “Pause” button indicates that the user is taking a break, and is temporarily not available for reading cases, even if still logged in.

A user who wished to read more than a fair share of desirable cases, without being known as a cherry-picker, might quickly switch back and forth between being logged in to different sites that he or she is affiliated with, and/or quickly switch back and forth between being logged in to different shifts, looking for more desirable cases. To try to prevent this from happening, cherry picking module 116 optionally monitors such behavior, and considers it evidence of cherry-picking. Alternatively or in addition, a user is deterred from switching repeatedly back and forth between different sites or different shifts because the site is informed about when the user is logged into the site and on what shift, and there is an expectation, on the part of the administrators of the site, that one of their affiliated radiologists will remain logged into the site on a single shift, for an extended period of time.

A summary table 308, on the left side of the display just under menu 306, lists the number of cases that the user is exposed to, sorted by level of urgency, and by other characteristics of the case. Each of the five rows of table 308 shows the number of cases at each level of urgency, with Level 1 (labeled “Stroke/Trauma”) at the top, followed by Level 2 (labeled “ED/ICU/STAT”), Level 3 (“IP”), Level 4 (“OP”), and Level 5 (“General”). The first column at the left, labeled “All,” shows the total number of cases in each level that the user is exposed to. The next column, labeled “My Site,” shows the number of cases at each level that come from the site that the user is logged in under. The next column, labeled “Asg. To Me,” shows the number of cases at each level that have been designated as to be assigned only to the user. As noted above, cases may be designated by the site that sends them for assigning only to a particular radiologist. As will be described below, a user can also designate a case as “Assigned to me,” and optionally such a designation can also be made by an administrator, or by another radiologist, who thinks that a case is especially suitable for one radiologist. The last column of table 308, at the right, labeled “My Sp,” shows the number of cases at each level that match the subspecialty of the user. In some embodiments of the invention, table 308, or of one or more rows or columns of table 308, is replaced or supplemented by graphical elements that convey the same information.

A worklist 310, listing cases that the user can choose for reading, is optionally the dominant feature, and is shown at the right side of the display. Each row of worklist 310 lists one case, and the cases are optionally grouped into sub-worklists by category of level of urgency, with Level 1 cases, typically labeled by the heading “Stroke/Trauma,” or a similar heading, listed on top, then Level 2 cases, Level 3 cases, Level 4 cases, and Level 5 cases, in that order, below the Level 1 cases, each sub-worklist with its own descriptive heading shown in a bar at the top, on the left side of the bar. Alternatively, only a single worklist, which may be a merged worklist combining the cases from the sub-worklists but displayed in a format not divided into sub-worklists, is displayed. A “merge” button 311, located just to the right of shift menu 306, optionally allows the user to switch between a merged view, where cases in different categories are merged together in a single list, and a sub-worklist view, where cases in different categories are displayed in separate sub-worklists as seen in FIG. 3.

The total number of cases in each level, the same numbers that are listed in the “All” column of table 308, is also listed in the bar at the top of each sub-worklist, just to the right of the descriptive heading. One or more measures of performance for each sub-worklist are optionally displayed in the heading bar. For example, in FIG. 3, the ratio of the median turn around time (TAT) for all cases, read during a past time interval, such as the past two hours, in the category of the sub-worklist, to the SLA time for the cases (assumed here to be the same for all cases in a given category), is shown after the words “Median TAT/SLA:” to the left of center of each bar. This median is optionally based on all cases in the system that were read during that time interval, not just on cases read by that user, and it provides a measure of the collective performance of the radiologists in the system. Optionally, the median turn around time is calculated at intervals short compared to the time interval on which the median is based, for example the median is calculated at 15 minute intervals, but is based on data from the past two hours. Alternatively, the median is based on only a subset of cases read during that time interval, for example only on radiologists with the same subspeciality as the user, or only on radiologists associated with the same site as the user. Optionally, the user can choose which of these “Median TAT/SLA” value is displayed in the bar, allowing the user to judge the recent performance of other radiologists in the same subspecialty or from the same site. Also, near the right end of the bar, the number of cases in that sub-worklist that are past their deadline is displayed in a red font, in a box further to the right, and the number of cases that are within 10 minutes of their deadline are displayed in an orange font further to the left. The red font and orange font are represented by a bold and non-bold font in FIG. 3. If one of these numbers is zero, then no number is displayed. These measures of performance, displayed in the bar at the top of each sub-worklist, provides an immediate indication of how well the radiologists as a whole are keeping up with the cases on that sub-worklist. Other measures of performance that are optionally displayed include the average time that it takes before a case is read, possibly normalized by the SLA time, and the percent of cases that are read by the deadline, or that are read by some specified time interval before the deadline. Alternatively, and particularly if the cases in worklist 310 are not grouped by sub-worklist but are displayed as a single merged worklist, these measures of performance for different sub-worklists are displayed in a table to the side of worklist 310, for example near table 308.

In some embodiments there are too many cases listed on the worklist to see all of them at once. In the view shown in FIG. 3, one of the sub-worklists, the one labeled “ED/ICU/STAT,” is shown expanded, with 9 listed cases visible, while the other sub-worklists are shown contracted, with only a few listed cases visible in each one. Alternatively, no cases are listed for contracted sub-worklists, but only the heading bar is shown. Optionally, for the expanded sub-worklists, or for one or more other sub-worklists as well, the heading bar gives the number of cases listed in that sub-worklist, as well as the total number of cases, for example the bar for the ED/ICU/STAT sub-worklist in FIG. 3 say “9/15” because 9 of the 15 cases in that sub-worklist are displayed. At the right side of each bar, there is a drop down menu that allows the user to expand or contract that sub-worklist. This can also be done by double-clicking on the corresponding row in table 308, or taking another user interface action on the corresponding row of table 308. When one sub-worklist is expanded, optionally any other sub-worklist that is already expanded contracts. If there is not enough room to display a list of all the cases on an expanded sub-worklist, then only the highest priority cases are listed in the display initially, and a user interface element such as a scroll bar or a “next page” button is optionally used to display a list of further cases. When a sub-worklist is expanded, the corresponding row in table 308 is optionally highlighted in some way, for example using a different color or style of font, and/or a different background color, to indicate that this sub-worklist is expanded.

Optionally, the cases listed in worklist 310 are filtered. The filtering is done, for example, by clicking on one of the headings of the columns in table 308. For example, clicking on the heading “My Site” displays only the cases from the user's site. Clicking on the heading “My Sp” displays only the cases matching the user's subspecialty. Clicking the heading “Asg. To Me” shows only the cases that have been reserved for assigning to the user. Clicking on “All” shows all of the cases, which is optionally the default. Optionally, more than one filter can be selected, for example selecting both the “My Site” and “My Sp” filters means that the only cases displayed will be those from the same site and with the same subspecialty as the user. Optionally, when a filter is selected, the statistical data shown on the heading bars is replaced by data based only on cases that match the filter. In all of these situations, the number of displayed cases might be limited by which sub-worklists are expanded and contracted, and by how many cases there is room to display at once, as described above.

Optionally, the cases in each sub-worklist are listed in order of priority, with the higher priority cases at the top. The order of priority is based, for example, on the time remaining till the deadline, with a case having higher priority if there is less time remaining till the deadline. Cases that are past their deadline are considered to have negative time to deadline, and cases that are further past their deadline would have higher priority. In some embodiments of the invention, the order of priority of cases within a sub-worklist is based not only on time to deadline, but on other factors as well, and depends on an index of importance. Particularly when the worklist is viewed in merged mode, with cases from different categories listed in a single order of priority, it may be advantageous for the order of priority to depend on an index of importance, rather than only on time to deadline, as described above under the heading “Setting an overall order of priority for cases from different categories.” An example of how such an index of importance is defined in given below in the description of FIG. 4.

Optionally, a fixed number N of the highest priority cases in each sub-worklist are recommended to the user, and the lower priority cases are not recommended. For example, in FIG. 3, N=4, at least for the ED/ICU/STAT worklist which is shown expanded. The first four cases are displayed in a dark, bold font, to indicate that they are recommended, while the remaining five cases shown in that sub-worklist are displayed in a gray or light-colored font, represented in FIG. 3 by a regular (non-bold) font, to indicate that they are not recommended. Since the other sub-worklists are shown contracted, with fewer than four cases visible on each one, all of the cases shown on the other sub-worklists are recommended, and are shown in a dark, bold font.

An optional “Indicators” column 312 of worklist 310 on the left includes indicators that can be used to display different kinds of information about each listed case. For each case, on the row where that case is listed, there are spaces for five indicators. The spaces are shown on FIG. 3 as empty squares. Furthest to the left for each row, there is a space for displaying a “Critical Results Notification” (CRN) indicator for that case, to indicate that an urgent medical condition was found in a preliminary reading of the case, and that the attending physician was or is being notified of this. For this and the other indicators displayed in column 312, the indicator is, for example, a font symbol such as a check mark or an X or a star, or the space is filled in with some color. Second from the left for each row, there is a space for an indicator that there exists a preliminary report for that case, for example a preliminary report prepared by a resident who is a trainee, or by a “night hawk” radiologist who is not an employee of the radiology practice and who is probably not trained in the subspecialty of the case but was on duty at night when the study was done. Optionally, user can display the preliminary report by clicking on this indicator. The user may want to look at the preliminary report, to help in interpreting the image that he is reading. Third from the left, in each row, is a space for an indicator that the case is “Assigned To Me,” meaning it is designated for assigning only to that user. Such a designation can be included when the case is sent from the site of origin, or a user can designate a case for assigning only to him/herself, or a supervisor or another radiologist can designate a case for assigning only to that user. But, as described above, certain kinds of cases, for example those with high enough level of urgency, or those sufficiently close to their deadlines for reading, will nevertheless be exposed to other radiologists, possibly to all available radiologists, to try to ensure that they are read by their deadlines. Fourth from the left in each row, is a space for an indicator that there are notes available about that case, for example notes entered by the technician who performed the study, or notes entered by a clerk at the request of a referring physician with relevant information. Optionally, the user can click on the indicator to display the notes. Fifth from the left in each row, there is a space to display a symbol to indicate a high degree of urgency for the case. Three such symbols are shown in FIG. 3, in the ED/ICU/STAT sub-worklist, though the symbols can be displayed for any cases in any worklist. In the second row of that sub-worklist in that space, there is a STAT symbol, in the form of an exclamation point, optionally in red, to indicate that the case is a STAT case. In the fourth row there is a “Hotline” symbol, an icon in the shape of a telephone receiver, to indicate that the case should be read immediately, not going through the normal procedure of waiting in a queue with other cases. A very short SLA time is optionally assigned to it, for example 15 minutes, and it is moved to the ED/ICU/STAT sub-worklist, in order to insure that it is read as soon as possible. In the last row of this sub-worklist, there is an “imaging analytics” symbol, which indicates that imaging analytics has found a feature that indicates an acute medical condition. If more than one of these symbols is applicable to a case, then optionally only of them is displayed. For example, the hotline symbol is displayed whenever it is applicable, the STAT symbol is displayed for a STAT case only if the hotline symbol is not applicable, and the imaging analytics symbol is displayed only if neither the STAT symbol nor the hotline symbol is applicable.

In a central portion of worklist 310, basic information about each case is displayed, each type of information in its own column. This information includes, for example, one or more of the patient's name, age, and sex, a unique patient identifier such as an ID number, a short description of the procedure, including the part of the body imaged and any use of contrast agents, the modality, the date and time at which the study was done, a symbol indicating the site that the case was sent from, and/or the time remaining till the reading deadline, in minutes.

In the STROKE/TRAUMA sub-worklist, the description of the procedure is optionally displayed in red for acute stroke cases, and black for trauma cases. This allows the user to quickly distinguish acute stroke cases from trauma cases, with acute stroke cases generally considered more urgent, while still including both acute stroke and trauma cases in Level 1, the highest level of urgency. In FIG. 3, this is indicated by the use of italic font for stroke cases, and non-italic font for trauma cases.

Listing the site of each case can allow users to see, at a glance, if one site is falling behind in reading their cases, since such a site will have many escalated cases. Optionally, escalated cases are displayed in a different font or color or with a symbol that makes it easy to see at a glance which cases are escalated. Furthermore, listing the site of each case may help users in choosing cases, since, for example, users may prefer to choose a case from a site they are associated with, rather than a case from a different site with a deadline that is only slightly earlier. The user's own site, the site the user is logged in under, indicated by the letter A in the “Site” column in FIG. 3, is optionally shown in bold and/or in a different color, and with an adjacent “My Site” indicator, an icon in the form of a building.

In the “Time Remaining” column, different font styles and/or colors are optionally used to indicate whether the case is before the first escalation time, between the first and second escalation times, after the second escalation time, or after the deadline. For example, the time remaining is displayed in black, non-bold, before the first escalation time, in bold orange between the first and second escalation time, and in bold red after the second escalation time, including after the deadline. Alternatively or additionally, a separate color is used for cases that are past their deadline. This allows the user to immediately identify which cases have been escalated, and the degree of escalation. It should be understood that these descriptions of font colors and styles to indicate escalated cases and the degree of escalation are only exemplary, and are optionally different in other system configurations and in other embodiments of the invention. In addition, other configurations and embodiments may have different numbers of escalation times.

Optionally, to choose a case for reading, the user activates a user interface element, for example right-clicking on the row for that case, or double-clicking on the row, to open a menu for the row. Optionally, the menu includes an item which allows the user to read the referring physician's diagnosis before deciding whether to choose the case, in addition to including an item for choosing the case. The menu also optionally includes an item for “Assign to Me,” reserving the case for the user, even if the user is not ready to read it yet. The menu also optionally includes an item allowing the user to reserve the case for a different radiologist whom the user thinks will be especially suitable for reading it. Even after a case has been reserved in that way for reading by the user or another specific radiologist, it may still be listed on the worklists of other radiologists after it has been escalated. The menu also optionally includes an item for the user to re-assign the case to a different category, or for advising an administrator that, in the opinion of the user, the case ought to be re-assigned to a different category. Optionally, the menu also includes items for accomplishing one or more functions of the PACS system for that case. Alternatively or additionally, any of the functions described here as included in this menu are implemented instead, or in addition, as additional action buttons located in “Actions” column 314 of the worklist, described below. Optionally, any of the functions described below for column 314 can be accomplished, instead or in addition, by choosing an item on this menu.

Once the user chooses the case, for example by selecting a “Choose” item from the menu, the software normally assigns the case to the user, unless another user has chosen the same case at almost the same time, as described above under the heading “Assignment of Cases.” When the case is assigned to the user, a new window is optionally opened, controlled by software included in the PACS, with provisions for displaying the radiology image or images to the user, optionally displaying the referring physician's diagnosis, and providing a user interface for the user to write, sign, and send a report on the case. Optionally, the user interface for writing the report includes at least an option for speech recognition, so that the report can be dictated, and the user can verify that it is correct. When the PACS software receives the report, it is stored on the PACS, and the referring physician and/or other medical personnel associated with the case are typically notified, so they can act on the report. Once the report has been sent, the new window is optionally closed, and the user can look at the worklist and choose a new case for reading.

In an “Actions” portion 314 of worklist 310 on the right side, there are action buttons for each case, that the user can use to take various actions. The buttons for each case are located on the row where the case is listed in the worklist. At the left side of portion 314, in each row, there is a “report to administrator” button, shown as an “R” in FIG. 3, to bring to the administrator's attention some special problem pertaining specifically to this case, that may need to be fixed before the case can be read, for example that the case is missing critical information, or that the case cannot be loaded. The user can select one of several fixed texts from a menu, and/or can enter free text, to create the report. Once the problem is confirmed, the case will be removed from all radiologist worklists and put on an administrator worklist, until the problem has been fixed.

The middle button in each row of portion 314 is an optional “break glass” button, shown as a “B” in FIG. 3, which the user can use in order to choose a non-recommended case for reading. The “break glass” button only appears for non-recommended cases. When the “break glass” button is used, a menu is optionally first displayed, from which the user can select a reason for choosing a non-recommended case, and the user's selection is logged by the system. Good reasons for selecting a non-recommended case might include the user's lack of expertise for reading any of the recommended cases displayed on the worklist, and the user's opinion that this non-recommended case should have higher priority for reading than any of the recommended cases, for clinical reasons. Optionally, the menu also has a “none of the above” choice that the user can pick, and/or the user can enter a reason manually. A user who too often uses the “break glass” option, especially without indicating a good reason, may be cherry picking. Even if the user indicated good reasons, the reasons are optionally reviewed by the user's supervisor or someone else, and if the reasons are found not to be valid, the user may be cherry picking. Additionally or alternatively, a user who has broken glass more than a threshold number of times in a specified time interval or while choosing a specified number of cases for reading, regardless of the reasons given, is suspected of cherry picking, on the supposition that a user would not normally have valid reasons for breaking glass so often. System administrators have several options for actions to take when a user is suspected of cherry picking, including one or more of the following: 1) the system software may sanction the user by exposing the user only to less desirable cases, cases that a cherry-picking user would not want to choose, for example cases that require relatively more time to read, and/or pay relatively less; 2) reprimand; 3) fine; 4) use of peer pressure.

On the right side of portion 314, for each case, there is an “additional information” button, shown as a plus sign in FIG. 3, for obtaining additional information about the case. This information includes, for example, the reason for the study, the number of images, the name and contact information for the ordering physician, and the account number, for example the patient's medical record number, and/or an accession number for that case. Optionally, the “additional information” button can also be used to access the referring physician's diagnosis.

At the left side of the display, below table 308, there are links 316 to other radiology cases that the user can view. These may include 1) recent cases that the user already read, that the user might want to look at again because they have some relevance to new cases that the user is reading; 2) peer review cases, that were read by other radiologists, that the user might want to review and comment on; and 3) teaching files that can be referred to by radiologists, particularly by radiologists who are being trained.

At the lower left of the display, there is an RVU Counter 318, displaying the RVUs accumulated so far by the user, for example for that day, and for the month to date. Alternatively, the RVU counter uses a different accounting unit, for example whatever accounting unit is used for calculating payments to the radiologists using system 100. RVU counter 318 also optionally shows the percentage difference between the RVUs accumulated by the user for the year to date, and an expected number of RVUs that the user should have accumulated, or an average number of RVUs that other users have accumulated, for the year to date. Optionally, the user's performance is displayed compared to an average for other radiologists in the system, or to an average for a group of radiologists such as those have the same subspecialty as the user, or those associated with the same site as the user.

At the top right, there is a search box 320 in which the radiologist may type a patient name; that will interrupt the normal workflow and open the case that is entered. This may be used, for example, if a referring physician enters the room and wishes to consult.

A chat icon 322 at the top right indicates there are unread messages for the user. Clicking on the chat icon opens a chat dialog window for reading or sending messages, for example to an attending physician for one of the cases on the worklist.

A series of tabs, not shown in FIG. 3, is optionally provided on top of the main display from which other functions not related to the major function of the orchestrator can be invoked.

### Example of how Worklists Change Over Time

An example will now be presented describing how the worklists of different radiologists change over time, and how the rules for recommending cases to radiologists can affect the efficiency of workflow, and can discourage cherry-picking, according to an exemplary embodiment of the invention. Suppose that, in each sub-worklist for each radiologist, only the first 4 cases, in order of priority, are recommended, and the other cases are not. It should be noted that the number “4” in this example is in general configurable, and can be set at the time of system installation, and modified by administrators as the system is optimized over time in response to its performance at a given installation. Suppose that there are 3 radiologists, Rad1, Rad2, and Rad3, each associated with a different site, in this example designated sites 1, 2, and 3, and each radiologist with a different area of specialization. Suppose that each site generates only cases that match one of the areas of specialization of the radiologists who are associated with them, and that these cases are all of the same level of urgency, a low enough level so that initially, each case is exposed only to radiologists who match the area of specialization of the case and are associated with the site that generated the case. After its second escalation time, any case that remains unread is exposed to all the radiologists. We assume that all cases have the same total time to deadline T_{SLA}.

Assume that the three sites each generate a conventional x-ray case, respectively called CR1A, CR2A, and CR3A, generated in that order at almost the same time, followed by a second conventional x-ray case, respectively called CRIB, CR2B, and CR3B, generated in that order at almost the same time, followed by a CT case, respectively called CT1, CT2, and CT3, followed by an MRI case, respectively called MR1, MR2, and MR3, all before the first escalation time of CR1A, CR2A and CR3A. In this example, the initial letters designate the imaging modality, and the numeric character designates the site at which the study was performed. Suppose the three radiologists all log in after MR1, MR2 and MR3 are generated, before the first escalation time of any of the cases; during this period visibility on a radiologist's worklist is restricted to cases that match both the site and the subspecialty of the radiologist. Because all of the cases have the same level of urgency, in this simplified example, the worklist for each radiologist consists of only one sub-worklist. Then the worklists for the three radiologists would be as shown in Table 2, with each column showing the worklist for one of the radiologists.

TABLE 2
Initial worklists

| Rad1 | Rad2 | Rad3 |
|---|---|---|
| CR1A | CR2A | CR3A |
| CR1B | CR2B | CR3B |
| CT1 | CT2 | CT3 |
| MR1 | MR2 | MR3 |

For each radiologist, only the cases that match their own area of specialization, and that come from the site they are associated with, are listed. (In this simplified example, all of the cases from each site match the subspecialty of the radiologist associated with that site.) All cases on the worklist of each radiologist are recommended, because there are only 4 cases on each sub-worklist, and this is indicated in Table 2 by showing the cases in bold font, rather than regular font. In some embodiments of the invention, recommended cases are distinguished from non-recommended cases by a different feature of the font, for example recommended cases are listed in a darker color font, such as black, and non-recommended cases are shown in a lighter color font, such as gray, in which case non-recommended cases may be referred to “grayed out.” The cases are listed in order of priority, with higher priority given to cases that have less time remaining to their deadline, and since all cases have the same total time to deadline T_{SLA}, the cases on each worklist are listed in the order that they were received.

Suppose all three radiologists prefer reading CT and MRI cases to conventional x-ray cases, because they are easier or more straightforward to read, or provide higher reimbursements, and each radiologist first chooses the CT case for reading, even though it is not the case with highest priority. Some radiologists in particular may tend to avoid reading conventional x-ray cases, because they do not feel comfortable reading them, even though conventional x-ray cases make up more than 50% of all radiology cases. Because, in this simplified example, both the sites and the subspecialties of the cases match the sites and subspecialties of the radiologists, the worklists do not change as the first escalation time passes. After time T1, the first escalation time for the other cases, the three sites each generate an ultrasound case, respectively called US1, US2, and US3, each with a subspecialty again matching the subspecialty of the radiologist associated with that site. The worklists then look as shown in Table 3.

TABLE 3
Worklists after time T1

| Rad1 | Rad2 | Rad3 |
|---|---|---|
| CR1A | CR2A | CR3A |
| CR1B | CR2B | CR3B |
| MR1 | MR2 | MR3 |
| US1 | US2 | US3 |

There are still only 4 cases listed on each sub-worklist, so all the cases on each worklist are recommended, as indicated by the bold font. At this time, the radiologists again ignore the higher priority conventional x-ray cases, and each chooses the more desirable MRI case to read.

After the second escalation time for the conventional x-ray cases, all the conventional x-ray cases, all of which are still not read, are exposed to all of the radiologists, since after the second escalation time, visibility is not limited to cases that match the radiologist in subspecialty or in site. The ultrasound cases, not yet past their second escalation time, are each only exposed to radiologists whose area of specialization they match. The worklists now look as shown in Table 4.

TABLE 4
Worklists after time T1 + T2

| Rad1 | Rad2 | Rad3 |
|---|---|---|
| CR1A | CR1A | CR1A |
| CR2A | CR2A | CR2A |
| CR3A | CR3A | CR3A |
| CR1B | CR1B | CR1B |
| CR2B | CR2B | CR2B |
| CR3B | CR3B | CR3B |
| US1 | US2 | US3 |

Because each radiologist now has 7 cases listed, only the first 4 cases on each sub-worklist are recommended, and the last three are not recommended, as indicated by being shown in regular (non-bold) font in Table 4. At this point, the radiologists cannot choose the ultrasound cases which they might prefer, because they are not recommended, unless they “break glass.” Although the radiologists can still choose these cases by using the break-glass option, they may be deterred from that course because their behavior is logged by cherry-picking module 116. Instead each radiologist is constrained to choose a conventional x-ray case, or wait and remain idle until the other radiologists read enough of the conventional x-ray cases, which will then disappear from the worklists, so that the ultrasound case is again recommended. This will mean that the radiologist who waits is working less efficiently, which would be reflected in his or her performance statistics. In addition, at least some of the radiologists might now choose cases that are not in their area of specialization, which may also cause a loss of efficiency in the work they do.

The negative situation shown in Table 4 is a result of the radiologists' earlier unbalanced reading, their choosing of more desirable lower priority cases ahead of less desirable higher priority cases. Note that the rule for recommending cases is configurable by system administrators. If the rule had been that only the first 2 cases on each sub-worklist are recommended, rather than 4 as in the preceding example, and if the radiologists did not want to choose non-recommended cases or to remain idle, then the radiologists would have been forced to choose conventional x-ray cases earlier on, and the situation shown in Table 4, where the highest priority cases of some radiologists do not match their area of specialization, might not have arisen. Then workflow might be more efficient if this rule were adopted. On the other hand, such a rule would give the radiologists less flexibility, and might be unnecessary if the radiologists as a whole were more cooperative, and had less of a tendency to engage in unbalanced reading, choosing only the most desirable cases. That very fact might motivate radiologists to behave in a more cooperative way.

### Exemplary Methods for Finding Index of Importance in Merged Worklist

When two or more sub-worklists, listing different categories of cases, are displayed showing a single order of priority, for example listed in order of priority in a single merged worklist, the order of priority is optionally found from an index of importance that depends on both the time to deadline and on the category and other characteristics of the cases, according to a predefined index of importance function. Table 5 shows the value of a component of the index of importance function as a function of time to deadline, and time past deadline, according to an exemplary embodiment of the invention, in which the index of importance function is the sum of a component that depends only on time to deadline, and a component that depends only on category. In this example, binning is used, and the value of the component depends only on which of several ranges the time to deadline is in. Alternatively, the value of the component is a continuous function of the time to deadline, with qualitatively similar behavior to that shown in Table 5. Using a continuous function has the potential advantage that it is unlikely that two cases will have the same index of importance, and cases in the same category, and with no differences in other characteristics that affect the index of importance, will likely have their order of priority based strictly on time to deadline. A potential advantage of binning is that there may be several cases with the same index of importance, and secondary considerations, such as a matching subspecialty or an association with the site of origin, can be taken into account in setting the order of priority between them.

TABLE 5
Value of component that depends on time to deadline

| Time to Deadline | | Component that depends on time to deadline |
|---|---|---|
| >2 | hours | 0 |
| 1-2 | hours | 1 |
| 30 min-1 hour | | 5 |
| 10 min-30 min | | 10 |
| 5-10 | min | 15 |
| 0-5 | min | 20 |

| | |
|---|---|
| <5 min past deadline | 25 |
| 5-10 min past deadline | 30 |
| 10-60 min past deadline | 35 |
| >60 min past deadline | 40 |

The values of the component given in Table 5 are roughly approximated, for example, by the continuous function 20[1−tanh(T/25)], where T is the time to deadline in minutes, and “tanh” is the hyperbolic tangent function.

Table 6 shows the value of the component that depends on category, for different categories of level of urgency. Note that, according to Table 5, the component that depends on time to deadline only changes by 30 from when the time to deadline is 30 minutes, until the time past deadline is more than 60 minutes, while according to Table 6, the difference in the value of the component that depends on category for acute stroke cases and for other cases is 30 or more. This means that, if the initial time to deadline for acute stroke cases is 30 minutes or less, and if the index of importance is the sum of the components given by Table 5 and Table 6 and does not include any other components, then acute stroke cases will always have an index of importance that is at least as high as that of any other cases, and higher than any cases other than trauma cases.

TABLE 6
Value of component that depends on category

| Category | Component that depends on category |
|---|---|
| Acute Stroke | 60 |
| Emergency Dept - Trauma | 30 |
| Emergency Dept - Other | 15 |
| ICU | 15 |
| Inpatient | 10 |
| Outpatient and other | 0 |

FIG. 4 shows a plot 400 of the index of importance function, as a function of time to deadline, for each of five categories of cases, labeled “Acute Stroke,” “Trauma,” “ICU”, “Inpatient,” and “Outpatient,” according to the embodiment shown in Tables 5 and 6. The “Emergency Dept—Other” category shown in Table 6 has the same index of importance as the “ICU” category. Plot 400 shows the time to deadline decreasing, going from left to right on a horizontal axis 402, with the time to deadline shown in minutes. The SLA time interval is assumed to be 30 minutes for acute stroke cases, 60 minutes for trauma cases, and 120 minutes for other cases. The index of importance function is shown on vertical axis 404. Negative values of time to deadline correspond to time after deadline.

Optionally, the index of importance function also includes other components that depend on other characteristics of the case. For example, in some embodiments of the invention, certain sites of origin, or certain attending physicians, receive preference in reading their cases, and this is optionally implemented by adding an extra component to the index of importance function for the preferred sites of origin and/or the preferred attending physicians. Optionally, the constant added for these other characteristics is relatively small, for example 10, so that acute stroke cases are still always, or almost always, have higher priority for reading than other cases.

The index of importance function need not have the same shape as a function of time to deadline for all categories and other characteristics, with only different additive constants, as in the index of importance described by Table 5 and Table 6 and shown in FIG. 4. In general, for cases having a given set of characteristics, including category, the index of importance function is some function of time to deadline. The functions are different for at least some different sets of characteristics, including different categories, and in some embodiments the functions have different shapes.

A property of the index of importance function as a function of time to deadline, for a given set of characteristics, is the range between maximum and minimum value that the function can have, for all times to deadline for which it is defined. The index of importance function is optionally not defined for times to deadline that are longer than the SLA time interval for cases with that set of characteristics, since the time to deadline will never be that long. Optionally the index of importance function is also not defined for very large negative times to deadline, since in practice a case may never remain unread that far past the deadline. The range between maximum and minimum value that the function can have is the smallest interval that includes all values that the function can have. This range is not necessarily equal to the range of the function, which is the set of values that the function can have, since the range may consist of only a finite set of discrete values, as is the case for the index of importance given by FIG. 4.

If this range between maximum and minimum values does not overlap for two different sets of characteristics, then cases having one of the sets of characteristics will always have a higher order of priority for reading than any cases with the other set of characteristics, regardless of the time to deadline. This is true, for example, of acute stroke cases and ICU cases according to the index of importance given by Tables 5 and 6. Acute stroke cases can have an index of importance ranging between 70 to 100, assuming that the SLA time interval for acute stroke cases is 30 minutes or less, and ICU cases can have an index of importance ranging between 15 and 55, so for any times to deadline, the index of importance is always higher for an acute stroke case, and it will always be higher in order of priority than any ICU case. If there is no overlap in the range between the maximum and minimum value of the function of time to deadline, for any two sets of characteristics, then the order of priority of reading cases will be sorted first by characteristics of the case, including category, and only among cases in a given category and a given set of other characteristics, will the cases be ordered by their time to deadline.

In some embodiments of the invention, there is at least some overlap in the range between maximum and minimum values that the index of importance function can have, for at least two different sets of characteristics of cases. If the index of importance function as a function of time to deadline is the same for these two sets of characteristics, for all times to deadline for which both functions are defined, then among cases with those sets of characteristics, the order of priority will depend only on time to deadline, regardless of which set of characteristics a case has. If the functions for the two sets of characteristics differ only in that one of them is shifted in time to deadline relative to the other, then the two functions can be considered to be the same, if time to deadline is replaced by an “effective time to deadline,” that differs from the actual time to deadline for cases having one of the sets of characteristics. In this case, the order of priority among cases having those sets of characteristics will depend only on the effective time to deadline of the case, regardless of which set of characteristics a case has.

However, if the two functions are not the same, and do not differ only by one of them being shifted in time relative to the other, then the order of priority will depend in a more complicated way on both the time to deadline, and on the characteristics of the case, such as the category of the case. This will be true if the range between maximum and minimum value of the function is different, but overlapping, for the two sets of characteristics. For example, for the index of importance given by FIG. 4, the range for ICU cases is 16 to 55, while the range for Out-patient cases is 1 to 40. For some values of time to deadline, an ICU case will have higher index of importance than an Out-patient case, but for other values of time to deadline, where the time to deadline is shorter for the Out-patient case, the Out-patient case will have higher index of importance than the ICU case. This can also be true if two functions have the same range between maximum and minimum value, but have different shapes. For some values of time to deadline, cases with one of the sets of characteristics will have higher index of importance, while for other values of time to deadline, cases with the other set of characteristics will have higher index of importance.

Optionally, the index of importance function for a given set of characteristics of the case always increases monotonically, with decreasing time to deadline. Even if the index of importance function is a binned function of time to deadline, the index of importance optionally always stays the same, or increases, as the time to deadline decreases. Alternatively, for at least some sets of characteristics, the index of importance function may first increase and then decrease with decreasing time to deadline. For example, as noted above in the section headed “Setting an overall order of priority for cases from different categories,” it may be advantageous to use such a non-monotonic index of importance for acute stroke cases, or for other types of cases that become less important to read once a critical time for treatment has passed.

Optionally, the index of importance function as a function of time to deadline, for a given set of characteristics of the cases, increases from its minimum value to a large part of the way to its maximum value, for example 50% or 70% or 90% of the way, over a limited range of values of time to deadline, for example over a range that is comparable to the SLA time interval. As the time to deadline becomes increasingly negative, the index of importance function reaches its maximum value, or becomes close to its maximum value, and does not further increase, or increases only slowly, as the time to deadline becomes more negative. This behavior of the index of importance function as a function of time to deadline may reflect the fact that for cases well past their deadline, the importance of reading them may depend more on their level of urgency, and other characteristics of the case, than on how much past their deadline they are. Optionally, the index of importance may go between 25% and 50% of the way from its minimum value to its maximum value, or between 50% and 75% of the way, or more than 75% of the way, as the time to deadline decreases by 25% of the SLA time interval, or by 50% of the SLA time interval, or by the SLA time interval, or by 2 times the SLA time interval, or by 4 times the SLA time interval.

### Using Feedback to Control Exposure Parameters

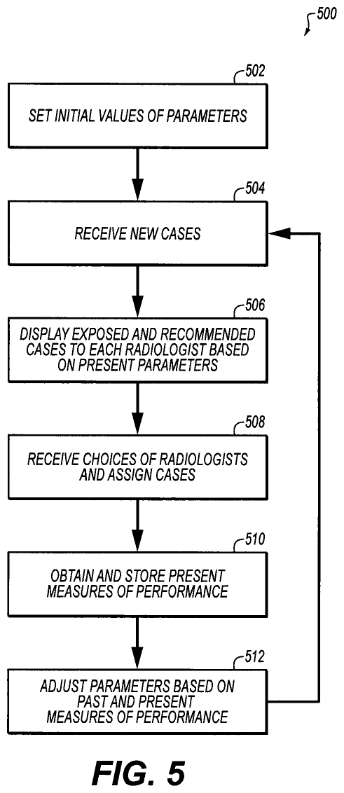

FIG. 5 shows a flowchart 500 for a method of using feedback from measures of performance of the system to dynamically control parameters that determine which cases are exposed and/or recommended to which radiologists, as discussed in the section headed, “Dynamically adjusting exposure parameters.” This kind of feedback can lead to improved system performance, including cases being read sooner on average, fewer cases remaining unread at their deadlines, a greater percentage of cases being read by radiologists with matching subspecialties and/or with an association with the site of origin, and more balanced reading between cases of different subspecialties, cases from different sites of origin, and cases in different categories of level of urgency. Possible exposure parameters that can be controlled in this way include the escalation times for different categories of cases, the number of cases that are recommended and the number of cases that are exposed, in each category, to each radiologist, and parameters controlling the dependence of index of importance on category of a case, in circumstances where the index of importance is used to set the order of priority of cases. Which control parameters are most effective to use is a question that might best be answered by experience. In general, using more control parameters might allow the system performance to be more optimized, but using more control parameters may make the feedback algorithm more complicated and difficult to implement.

At 502, the control parameters are set to initial values that are generally expected to work well. At 504, new cases are received. At 506, the currently unread cases are displayed to the radiologists, according to criteria for exposing and recommending cases set by the present values of the control parameters. At 508, the radiologist choose cases for reading, from among the cases they are exposed to, the choices of the radiologists are received by the system, and cases are assigned to the radiologists based on their choices. At 510, measures of performance, based on the present situation and the recent past, are obtained and stored in memory. Optionally, the measures of performance are obtained using time windows, optionally time windows that overlap between consecutive calculations of the measures of performance.

At 512, the values of one or more control parameters are adjusted, based on the present and/or past values of the performance parameters. Optionally, the values of the control parameters are changed by a magnitude and in a direction that is expected to optimize an overall measure of performance. An expected relationship between the control parameters and the measures of performance is optionally based on first principles modeling, and/or on past experience with the values of different measures of performance that result from setting the control parameters at different values. The new values of the control parameters are calculated from the old values, from the measures of performance, and optionally also from a definition of an overall measure of performance that is being optimized, using any feedback algorithm known in the art, taking into account the expected relationship between the control parameters and the measures of performance. Optionally, the time histories of the measures of performance and the control parameters are recorded, for later examination by administrators to see how the control parameters affect the measures of performance, and how the feedback algorithm can be improved.

Optionally, the control parameters are adjusted separately for different subspecialties, different sites of origin, and/or different categories of cases, particularly if the measures of performance indicate that one or more subspecialties, sites of origin, or categories is being neglected.

After the values of the control parameters have been adjusted, new cases are received again at 504, and the new values of the control parameters are used for exposing and recommending cases at 506.

### Use of Imaging Analytics

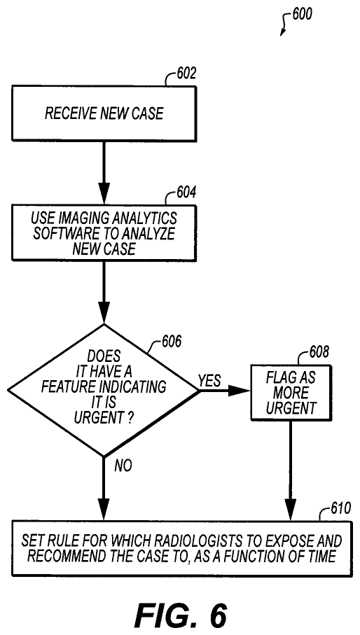

FIG. 6 shows a flowchart 600 for a method of using imaging analytics to determine the urgency of a case, according to an exemplary embodiment of the invention. At 602, a new case is received. At 604, one or more images from the new case are analyzed using imaging analytics software, to look for one or more features that would indicate that the case might be more urgent for reading than was apparent from clinical symptoms at the time the study was done. For example, the feature is free air in the abdomen, which can indicate a perforated bowel that requires immediate treatment, or free air in the thorax, or a hemorrhage or an aneurysm that may not have been previously suspected. Alternatively, the study was done as part of a screening of a large number of people to look for a medical condition that only a small fraction of the people screened are likely to have, and the feature is an indication that the medical condition is present.

At 606, a decision is made as to whether the feature has been found in the image or not. If the feature has been found, then the case is flagged as more urgent, at 608. At 610, a rule is applied for which radiologists the case will be exposed and recommended to, as a function of time. This rule will depend on whether the case has been flagged as more urgent at 608. For example, if the case has been flagged as more urgent, then it may be exposed or recommended to a greater number of radiologists, at a given time, than if it were not flagged as more urgent.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

It is expected that during the life of a patent maturing from this application many relevant medical imaging modalities will be developed and the scope of the term modality is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1.-58. (canceled)

59. A method of automatically assigning each of a plurality of medical imaging cases for reading by one of a plurality of readers, the method comprising:
a) receiving the medical imaging cases by a computer system, from one or more sites;
b) the computer system including information about each case, at different times, on worklists that the computer system displays on workstations of one or more readers who are allowed to choose the case for reading at that time, the worklists indicating to each of those readers if they are encouraged to choose the case for reading at that time;
c) the computer system receiving information on choices of cases by the readers; and
d) the computer system assigning cases to workstations of readers who choose them;
wherein for at least some of the cases, initially only a portion of the readers are allowed or encouraged to choose the case, but over time, as the case becomes more urgent to read if it is still not read, the case is escalated a first time by the computer system adding one or more other readers to the readers who are allowed or encouraged to choose the case, and over further time, if the case is still not read, the case is escalated at least one additional time by the computer system adding one or more other readers to the readers who are allowed or encouraged to choose the case.

60. The method according to claim 59, wherein each reader has a profile accessible to the computer system indicating the reader's characteristics, and wherein initially, readers are allowed to choose a case, or are encouraged to choose a case, only if they there is a subspecialty which matches the case indicated on the reader's profile, or only if there is a relationship with a site of origin of the case indicated on the reader's profile, or both, and when the case is escalated, the added readers have a different subspecialty, or do not have a relationship with the site of origin, or both, indicated on their profiles.

61. The method according to claim 60, wherein, when a case is escalated one of the times, there is no relationship with the site of origin indicated on the added readers' profiles, but there is a subspecialty which matches the case indicated on the added readers' profiles, and when the case is escalated at a later one of the times, there is no subspecialty which matches the case indicated on the added readers' profiles.

62. The method according to claim 59, also comprising:
a) for each case, the computer system determining an urgency for reading the case as a function of time; and
b) for each case, the computer system determining one or more criteria for which of the readers will be allowed to choose the case for reading, and for which readers will be encouraged to choose the case for reading, as a function of time, based at least in part on the urgency for reading the case as a function of time;
wherein including information on each case on worklists comprises including information on each case on worklists displayed on the workstations of the readers who are allowed to choose it at that time according to the criteria, indicating to the readers if they are encouraged to choose the case at that time according to the criteria.

63. The method according to claim 62, wherein determining criteria for which readers will be encouraged to choose a case at a given time comprises determining criteria for setting a relative priority of a case that a reader is allowed to choose, compared to other cases that the reader is allowed to choose, based at least in part on the urgency for reading the case at that time, and including information about each case on worklists of one or more readers who are allowed to choose it comprises indicating on the worklist of each of those readers the relative priority of the case.

64. The method according to claim 63, wherein determining criteria for determining a relative priority of a case for a reader comprises determining criteria for whether or not the case is recommended for the reader.

65. The method according to claim 62, wherein each reader has a profile accessible to the computer system indicating the reader's characteristics, and the criteria depend on whether or not a reader's profile indicates a subspecialty that matches the case.

66. The method according to claim 62, wherein determining criteria comprises determining criteria that depend on one or more of work shift, time of day, day of the week, and whether it is a holiday.

67. The method according to claim 62, also comprising analyzing images from the medical imaging cases by imaging analytics circuitry to determine a presence or absence of one or more features that indicate a higher degree of urgency for reading the case, wherein determining an urgency for reading the case as a function of time comprises determining a higher degree of urgency at a given time for a case in which the one or more features are present than for a case with otherwise identical characteristics received at the same time for which the one or more features are absent.

68. The method according to claim 67, wherein, according to the criteria, for at least some times and at least some characteristics of cases, more readers would be allowed or encouraged to choose a case in which the one or more features are present, than a case with otherwise identical characteristics received at the same time, in which the one or more features are absent.

69. The method according to claim 67, wherein the one or more features comprise free air in a patient's abdominal cavity or thorax.

70. The method according to claim 62, wherein the cases received pertain to a plurality of different medical conditions, and determining an urgency for reading the case comprises determining an urgency that depends at least in part on the medical condition that the case pertains to.

71. The method according to claim 59, wherein including information about each case on worklists displayed on workstations of one or more readers comprises including information about a case that was escalated on a same worklist on a workstation of one of the readers who was added when the case was escalated, as information about a case that was not escalated.

72. The method according to claim 71, comprising the computer system determining a single order of priority for all cases for which information is included on the worklist.

73. The method according to claim 71, comprising the computer system arranging cases for which information is included on the worklist into two or more sub-worklists, each sub-worklist including a different category of cases, and determining a separate order of priority for each sub-worklist.

74. The method according to claim 59, wherein each reader has a profile indicating the reader's characteristics, accessible to the computer system, the method also comprising the computer system displaying together with the worklist on the workstations of at least some of the readers how many unread cases match each of a plurality of different subspecialties, including subspecialties that are not indicated on the profile of that reader, or how many unread cases come from each of a plurality of different sites of origin, or both.

75. The method according to claim 59, further comprising:
a) logging in to the computer system by one or more readers, wherein the computer system displaying the worklists comprises displaying the worklists on the workstations of the readers who are logged in; and
b) setting criteria that determine which cases are included on the worklists displayed on the workstations of the readers who are logged in, allowing the readers to choose those cases for reading, wherein the criteria in at least some circumstances allow a case to be included on a worklist displayed on a workstation of a reader who is not associated with a site that the case is from, together with cases from a site that the reader is associated with.

76. A method of automatically assigning each of a plurality of medical imaging cases for reading by one of a plurality of readers, the method comprising:
a) receiving the medical imaging cases by a computer system;
b) for each case, the computer system determining an urgency for reading the case as a function of time;
c) for each case, the computer system determining one or more criteria for which of the readers will be allowed to choose the case for reading, and for each reader who is allowed to choose the case, the computer system determining a relative priority of the case compared to other cases the reader is allowed to choose, based at least in part on the urgency for reading the case;
d) the computer system including information about each case in a worklist that the computer system displays on a workstation of each of one or more readers who are allowed to choose it for reading, the information indicating to the readers the relative priority of different cases they are allowed to choose;
e) the computer system receiving information on choices of cases by the readers;
f) the computer system assigning cases to readers who choose them; and
g) the computer system keeping track of a measure of an extent to which different readers choose cases of lower priority than other cases they are allowed to choose.

77. A computer system for automatically assigning each of a plurality of medical imaging cases for reading by one of a plurality of readers, the system comprising:
a) a receiving module configured to receive medical imaging cases from one or more sites;
b) a workstation used by each reader;
c) a reader I/O module configured to include information about different sets of the received medical imaging cases in worklists that the computer system displays on the workstations of different readers, to indicate that a reader is encouraged to choose a case for which information is displayed, to receive information about the readers' choices of cases for reading, and to assign cases to the workstations of the readers; and
d) a computer running software configured to:
1) include information about each case, at different times, on worklists that the computer system displays on the workstations of one or more readers who are allowed to choose it for reading at that time, the information indicating to each of those readers if they are encouraged to choose the case for reading at that time;
2) receive information about choices of cases by the readers; and
3) assign cases to the workstations of readers who choose them;
wherein the software is configured so that, for at least some of the cases, initially only a portion of the readers are allowed or encouraged to choose the case, but over time, as the case becomes more urgent to read if it is still not read, the case is escalated a first time by the computer system adding one or more other readers to the readers who are allowed or encouraged to choose the case, and over further time, if the case is still not read, the case is escalated at least one additional time by the computer system adding one or more other readers to the readers who are allowed or encouraged to choose the case.

78. A method of automatically assigning each of a plurality of medical imaging cases for reading by one of a plurality of readers, the method comprising:
a) receiving the medical imaging cases by a computer system;
b) for each case, the computer system determining an urgency for reading the case as a function of time, using a rule that depends on one or more characteristics of the case;
c) for each case, the computer system determining one or more criteria, depending on characteristics of the case, characteristics of readers, and the urgency for reading the case, for which of the readers will be allowed to choose the case for reading, and for each reader who is allowed to choose the case, whether the reader is encouraged to choose the case, and any priority the case is given for each reader who is allowed to choose the case, over other cases the reader is allowed to choose;
d) the computer system including information about each case in a worklist that the computer system displays on a workstation of each of one or more readers who are allowed to choose it for reading, the information indicating to the readers any relative priority of different cases they are allowed to choose;
e) the computer system receiving information on choices of cases by the readers;
f) the computer system assigning cases to readers who choose them;
g) the computer system evaluating one or more measures of performance of reading the cases;
h) the computer system using the measures of performance to determine a new value of one or more parameters defining the rule or the criteria that is predicted to improve an overall level of the performance; and
i) the computer system applying the new value of the one or more parameters to adjust the rule or the criteria for new medical imaging cases received by the computer.

79. A method of automatically assigning each of a plurality of medical imaging cases for reading by one of a plurality of readers, the method comprising:
a) receiving the medical imaging cases by a computer system, from one or more sites;
b) analyzing images from the medical imaging cases by imaging analytics circuitry to determine a presence or absence of one or more features that indicate a higher degree of urgency for reading the case;
c) for each case, the computer system determining an urgency for reading the case as a function of time, including determining a higher degree of urgency at a given time for a case in which the one or more features are present than for a case with otherwise identical characteristics received at the same time for which the one or more features are absent;
d) for each case, the computer system determining one or more criteria for which of the readers will be allowed to choose the case for reading, and for which readers will be encouraged to choose the case for reading, as a function of time, based at least in part on the urgency for reading the case as a function of time;
e) the computer system including information about each case, at different times until it is chosen for reading, on worklists that the computer system displays on workstations of one or more readers who are allowed to choose it for reading at that time according to the criteria, the worklists indicating to each of those readers if they are encouraged to choose the case for reading at that time according to the criteria;
f) the computer system receiving information on choices of cases by the readers; and
g) the computer system assigning cases to workstations of readers who choose them.
